(19) United States
(12) Patent Application Publication
(10) Pub. No.: US 2023/0101896 A1
(43) Pub. Date: Mar. 30, 2023

(54) Enhanced Detection of Target Nucleic Acids by Removal of DNA-RNA Cross Contamination

(71) Applicant: Mission Bio, Inc., South San Francisco, CA (US)

(72) Inventor: Daniel ` Mendoza, South San Francisco, CA (US)

(21) Appl. No.: 17/801,017

(22) PCT Filed: Feb. 21, 2021
(86) PCT No.: PCT/US2021/018945

§ 371 Date: Aug. 19, 2022

Related U.S. Application Data
(60) Provisional application No. 62/979,651, filed on Feb. 21, 2020.

(51) Int. Cl. C12Q 1/6848

(52) U.S. Cl. CPC C12Q 1/6848

(57) ABSTRACT

Cleavable primers are incorporated into single cell analysis workflows to reduce and/or eliminate misprimed nucleic acid amplicons. Specifically, cleavable primers can introduce restriction endonuclease cleavage sites into misprimed nucleic acid amplicons. For example, cleavable primers can introduce a restriction endonuclease cleavage site into an amplicon comprising DNA misprimed by an RNA primer. As another example, cleavable primers can introduce a restriction endonuclease cleavage site into an amplicon comprising cDNA misprimed by a DNA primer. Such amplicons can then be cleaved by a restriction endonuclease to remove them from identification and association in subsequent nucleic acid sequencing.

### CROSS REFERENCE

This application claims the benefit of and priority to U.S. Provisional Patent Application No. 62/979,651 filed Feb. 21, 2020, the entire disclosure of which is hereby incorporated by reference in its entirety for all purposes.

### BACKGROUND

A challenge in high throughput single-cell multi-omic analysis arises where cross-contamination occurs between different types of nucleic acids (e.g., between RNA and DNA) in the single cell analysis. For example, primers added to analyze RNA and DNA can result in primer byproducts and mispriming of DNA by the reverse transcription primers and mispriming of RNA by DNA primers. These primer byproducts and misprimed nucleic acids can be problematic as they can result in erroneous sequence reads and/or inaccurate characterization of individual cells. In other words, in scenarios such as multi-omic (e.g., RNA and DNA) single cell analysis, the presence of primer byproducts and/or misprimed nucleic acids results in qualitatively poor analysis of single cells.

### SUMMARY

The disclosure generally relates to methods and apparati for single-cell analysis through the implementation of post-amplification reaction clean up using restriction endonucleases. In various embodiments, the restriction endonucleases selectively cleave misprimed amplicons, preventing their inclusion in sequencing reads. Altogether, the implementation of restriction endonucleases for reaction clean-up represents an improved single-cell analysis workflow which, in particular embodiments, involves a multi-omic single-cell analysis workflow (e.g., DNA and RNA analysis), which achieves improved sequence read metrics (e.g., improved percentage of reads after trimming, improved percentage of mapped reads, and/or improved percentage of reads with a valid cell barcode).

Disclosed herein is a method for determining the presence or absence of a target nucleic acid from a single cell, the method comprising: obtaining a cell lysate from a single cell in a reaction mixture droplet; adding oligonucleotide primers to the reaction mixture droplet, wherein the oligonucleotide primers comprise restriction endonuclease cleavage sites; catalyzing a nucleic acid amplification reaction using the primers to produce an amplicon from a target nucleic acid molecule, if present, in the single cell; adding restriction endonucleases to the reaction mixture droplet and incubating to cleave, if present, nucleic acid amplicons that have been misprimed by the oligonucleotide primers; sequencing the remaining amplicons; and determining the presence or absence of the target nucleic acid from the single cell based on the presence or absence of the target sequence in the determined sequences of the amplicons.

In some embodiments, the nucleic acid amplicons comprise misprimed DNA that have been misprimed by oligonucleotide primers. In some embodiments, the target nucleic acid sequence is complementary to an mRNA that corresponds to the DNA, wherein the DNA comprises a restriction endonuclease cleavage sequence. In some embodiments, the oligonucleotide primers are DNA primers. In some embodiments, one of the oligonucleotide primers comprises a forward primer that is complementary to the target nucleic acid sequence. In some embodiments, the forward primer is a gene specific primer. In some embodiments, one of the oligonucleotide primers further comprises a constant region. In some embodiments, a restriction endonuclease cleavage site is located between the constant region and the forward primer.

In some embodiments, the DNA is genomic DNA. In some embodiments, the DNA is an oligonucleotide sequence corresponding to an affinity reagent. In some embodiments, the oligonucleotide sequence was previously conjugated to the affinity reagent. In some embodiments, one of the oligonucleotide primers comprises a random primer that is complementary to the target nucleic acid sequence. In some embodiments, the one of the oligonucleotide primers further comprises a constant region. In some embodiments, a restriction endonuclease cleavage site is located between the constant region and the random primer.

In some embodiments, the nucleic acid amplicons comprise misprimed cDNA generated from RNA of the single cell, wherein the misprimed cDNA have been misprimed by oligonucleotide primers. In some embodiments, the target nucleic acid sequence is a gDNA that corresponds to the misprimed cDNA, wherein the misprimed cDNA comprises one or more restriction endonuclease cleavage sequences. In some embodiments, the oligonucleotide primers are DNA primers. In some embodiments, one of the oligonucleotide primers comprises a forward primer that is complementary to the target nucleic acid sequence. In some embodiments, the forward primer is a gene specific primer. In some embodiments, the one of the oligonucleotide primers further comprises a constant region. In some embodiments, a restriction endonuclease cleavage site is located between the constant region and the forward primer.

In some embodiments, the restriction endonucleases have low frequencies of cleavage sites in gDNA. In some embodiments, the restriction endonucleases have higher frequencies of cleavage sites in introns than in exons. In some embodiments, the restriction endonucleases have 50% or more, 60% or more, 70% or more, 80% or more, or 90% or more higher frequency of cleavage sites in introns than in exons. In some embodiments, the restriction endonucleases have a high frequency of cleavage sites in the human genome in CpG islands. In some embodiments, the restriction endonucleases have 75% or more, 80% or more 85% or more, or 90% or more cleavage sites in the human genome in CpG islands. In some embodiments, wherein the restriction endonucleases are selected from AscI, NotI, AfeI, and SapI.

In some embodiments, the determination of the presence or absence of the target nucleic has increased specificity compared to detection in samples where restriction endonucleases are not added. In some embodiments, the determination of the presence or absence of the target nucleic acid has 50% or more, 60% or more, 70% or more, 80% or more, or 90% or more increased specificity compared to detection in samples where restriction endonucleases are not added. In some embodiments, the determination of the presence or absence of the target nucleic has a decreased incidence of false positives compared to detection in samples where restriction endonucleases are not added. In some embodiments, the determination of the presence or absence of the target nucleic acid has 50% or more, 60% or more, 70% or more, 80% or more, or 90% or more decreased incidence of false positives compared to detection in samples where restriction endonucleases are not added.

In some embodiments, the method further comprises adding oligonucleotide barcodes to the reaction mixture droplet, the oligonucleotide barcodes specific for the reaction mixture droplet. In some embodiments, the sequencing comprises identification of target nucleic acids by identification of the oligonucleotide barcode. In some embodiments, the sequencing comprises sequencing a nucleic acid lacking an oligonucleotide barcode; and removing the sequenced nucleic acid for further analysis. In some embodiments, the nucleic acid lacking the oligonucleotide barcode was previously a nucleic acid amplicon that was misprimed by an oligonucleotide primer and cleaved by a restriction endonuclease.

In some embodiments, the reaction mixture droplet is an aqueous solution, an aqueous emulsion in oil, or an aqueous suspension in oil. In some embodiments, the reaction mixture droplet comprises a DNA-modifying enzyme for synthesizing cDNA from RNA, DNA extension, hybridization, capture, or ligation. In some embodiments, obtaining the cell lysate from the single cell in the reaction mixture droplet comprises exposing the single cell in the reaction mixture droplet to a protease. In some embodiments, the protease is proteinase K.

BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG.) 1A shows an overall system environment for analyzing cell(s) through a single cell workflow analysis, in accordance with an embodiment.

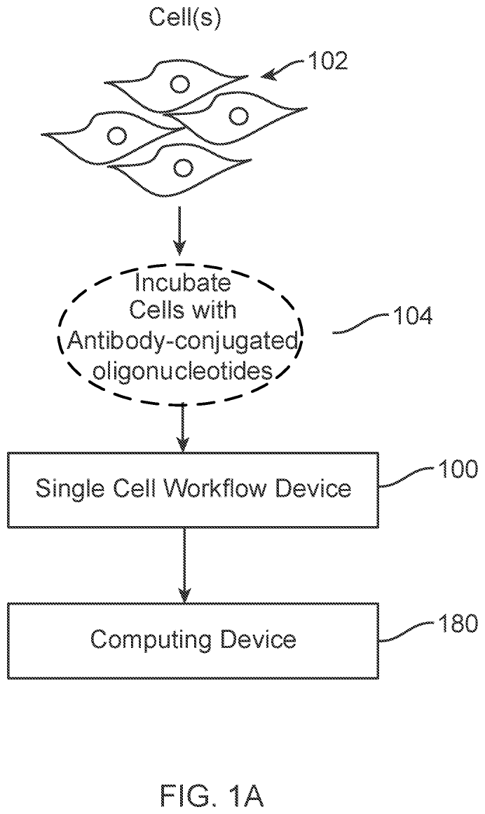

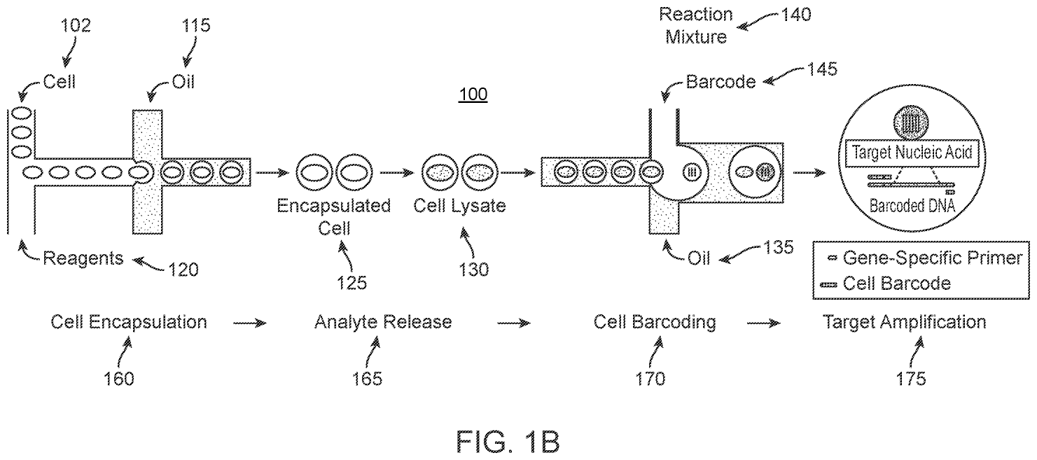

FIG. 1B depicts a single cell workflow analysis to generate amplified nucleic acid molecules for sequencing, in accordance with an embodiment.

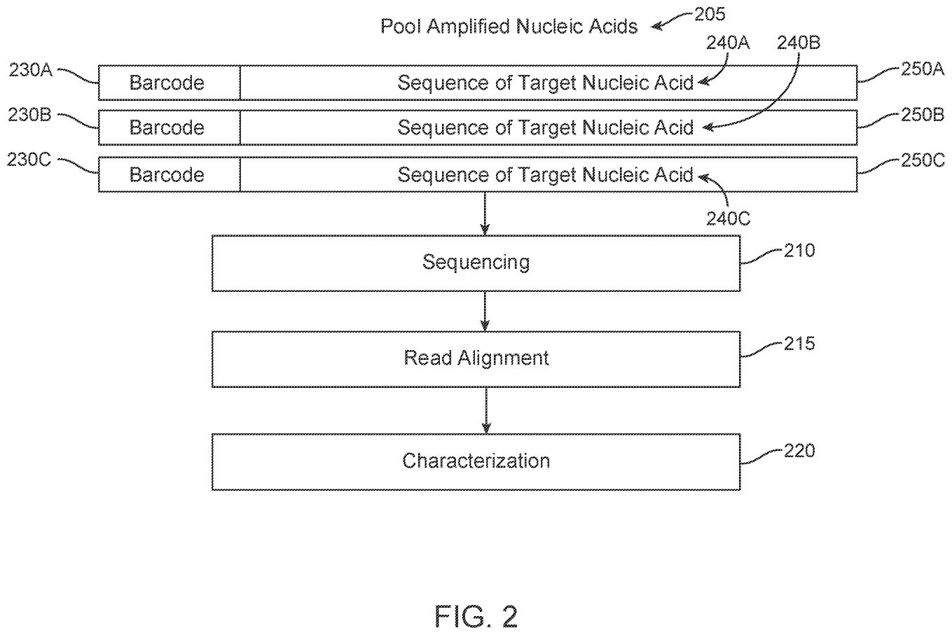

FIG. 2 is a flow process for analyzing nucleic acid sequences derived from analytes of the single cell, in accordance with an embodiment.

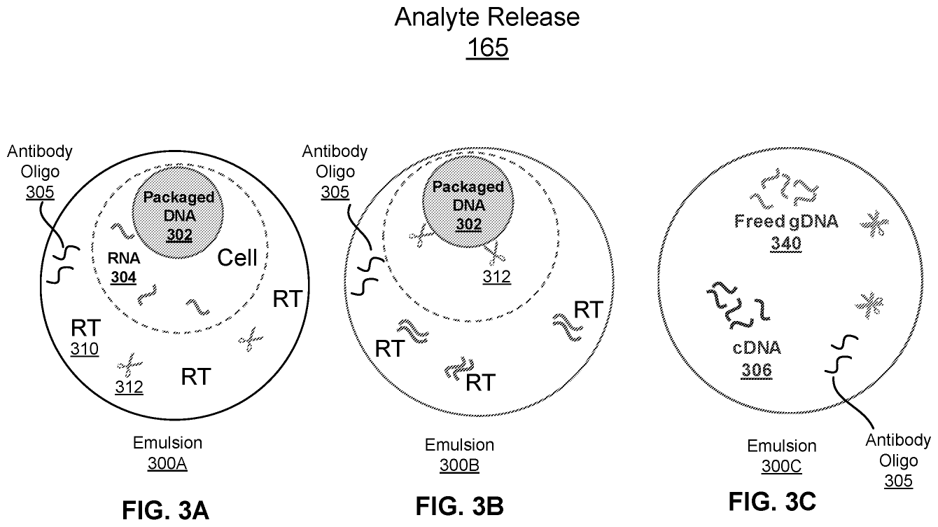

FIGS. 3A-3C depict the processing and releasing of analytes of a single cell in a droplet, in accordance with an embodiment.

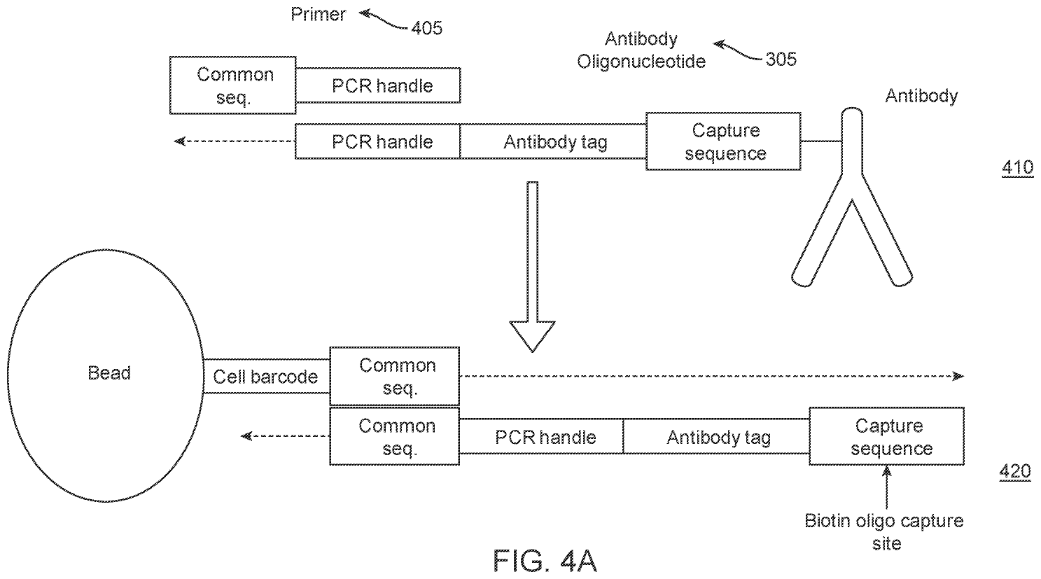

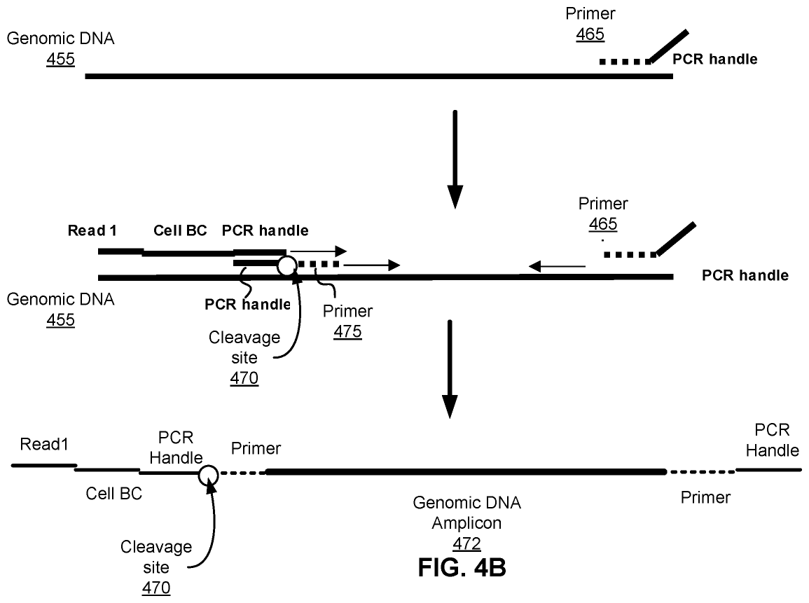

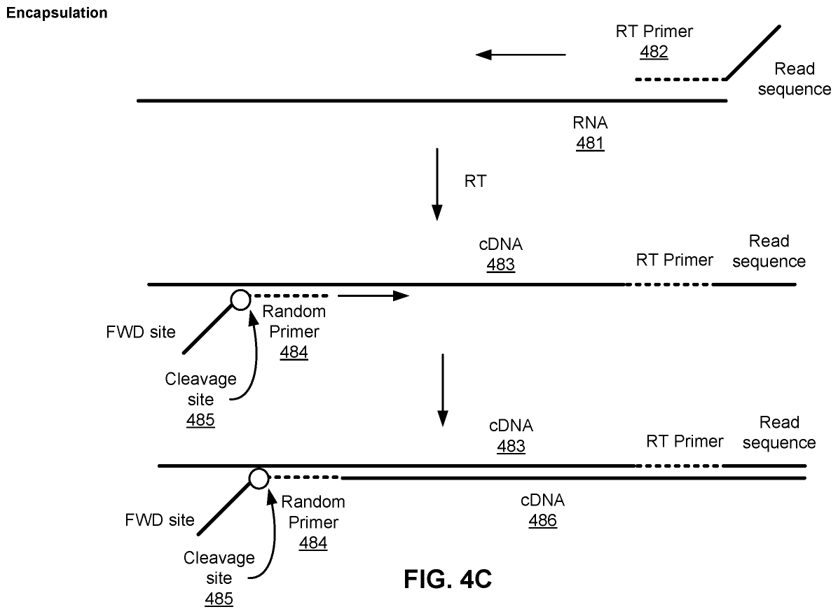

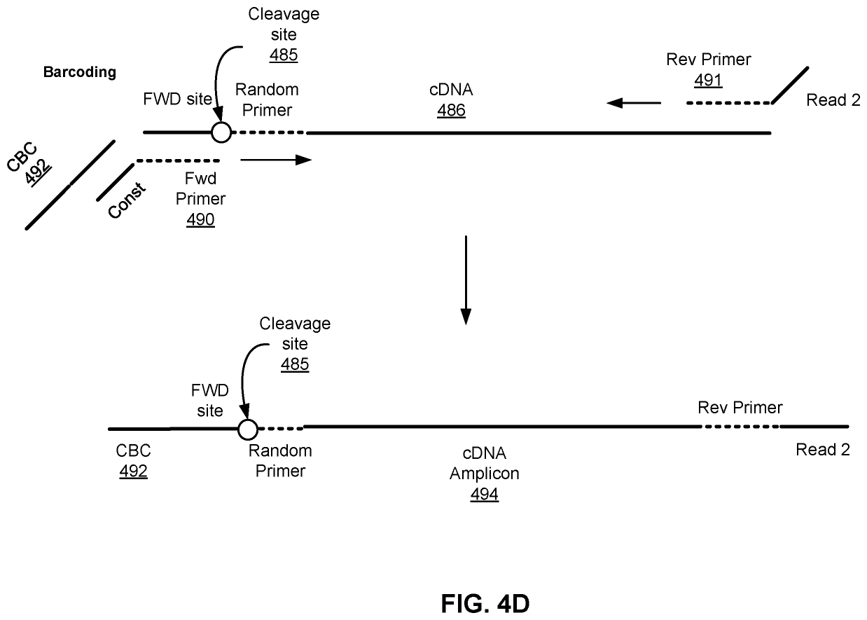

FIG. 4A illustrates the priming and barcoding of an antibody-conjugated oligonucleotide, in accordance with an embodiment.

FIG. 4B depicts the amplification and barcoding of nucleic acids derived from gDNA, in accordance with an embodiment.

FIGS. 4C and 4D depict the amplification and barcoding of nucleic acids derived from RNA, in accordance with an embodiment.

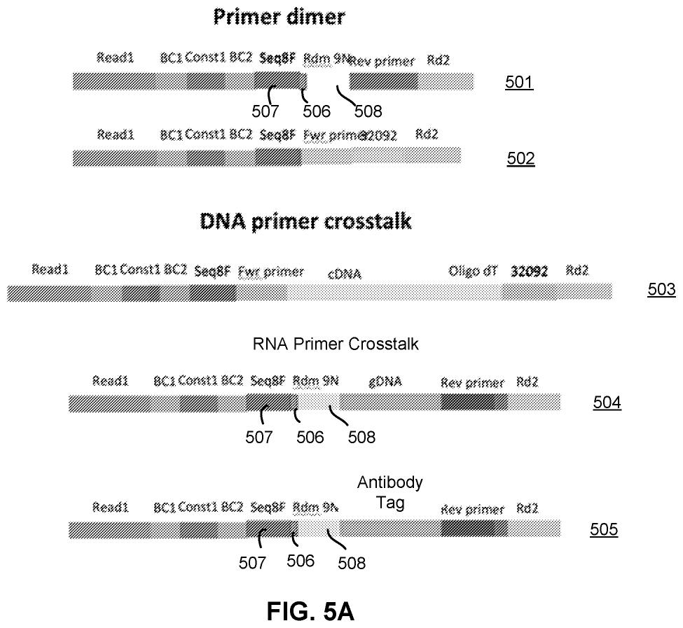

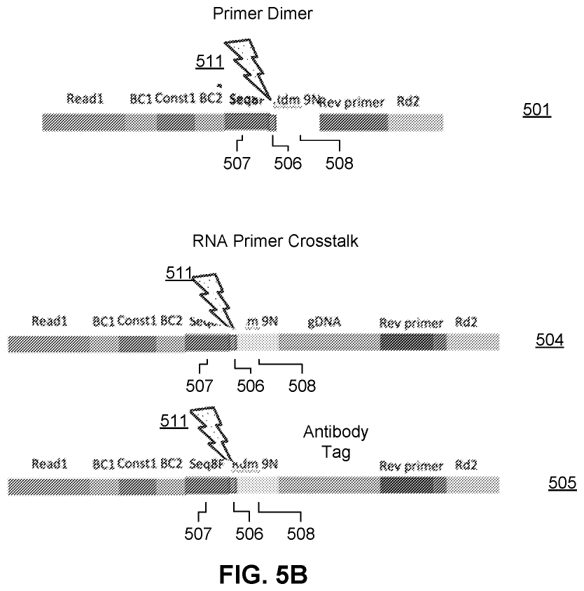

FIG. 5A depicts the nucleic acid amplicons arising from RNA primers including primer dimers, misprimed gDNA and misprimed antibody tag DNA.

FIG. 5B depicts the cleavage of misprimed nucleic acid amplicons arising from RNA primers, in accordance with the embodiment shown in FIG. 5A.

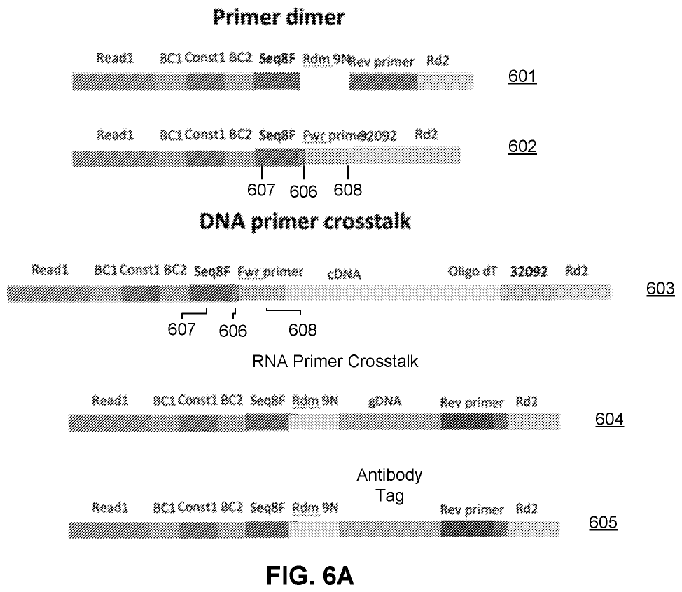

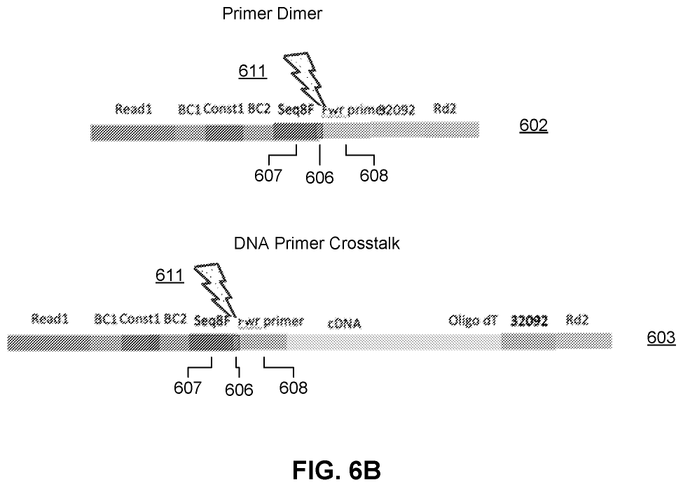

FIG. 6A depicts the nucleic acid amplicons arising from DNA primers including primer dimers and misprimed cDNA.

FIG. 6B depicts the cleavage of misprimed nucleic acid amplicons arising from DNA primers, in accordance with the embodiment shown in FIG. 6A.

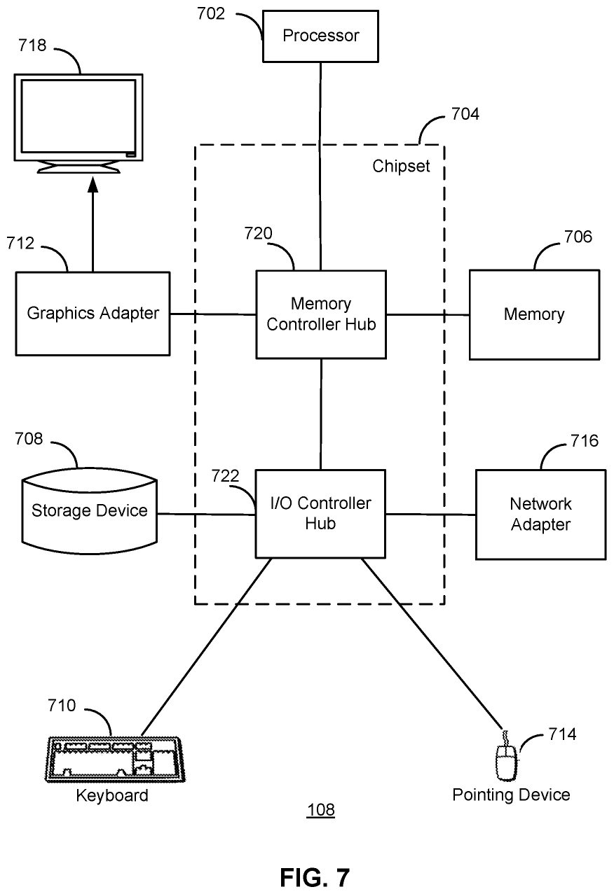

FIG. 7 depicts an example computing device for implementing system and methods described in reference to FIGS. 1-6.

### DETAILED DESCRIPTION

Definitions

Terms used in the claims and specification are defined as set forth below unless otherwise specified.

The term “subject” or “patient” are used interchangeably and encompass an organism, human or non-human, mammal or non-mammal, male or female.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “analyte” refers to a component of a cell. Cell analytes can be informative for characterizing a cell. Therefore, performing single-cell analysis of one or more analytes of a cell using the systems and methods described herein are informative for determining a state or behavior of a cell. Examples of an analyte include a nucleic acid (e.g., RNA, DNA, cDNA), a protein, a peptide, an antibody, an antibody fragment, a polysaccharide, a sugar, a lipid, a small molecule, or combinations thereof. In particular embodiments, a single-cell analysis involves analyzing two different analytes such as RNA and DNA. In particular embodiments, a single-cell analysis involves analyzing three or more different analytes of a cell, such as RNA, DNA, and protein.

In some embodiments, the discrete entities as described herein are droplets. The terms “emulsion,” “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible phase carrier fluid. Thus droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, reaction mixture, and a variety of other components. The term emulsion may be used to refer to an emulsion produced in, on, or by a microfluidic device and/or flowed from or applied by a microfluidic device.

“Complementarity” or “complementary” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See, e.g., Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

The terms “amplify,” “amplifying,” “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). In some embodiments, the amplification reaction includes an isothermal amplification reaction such as LAMP. In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

Any nucleic acid amplification method may be utilized, such as a PCR-based assay, e.g., quantitative PCR (qPCR), or an isothermal amplification may be used to detect the presence of certain nucleic acids, e.g., genes of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

“Forward primer binding site” and “reverse primer binding site” refer to the regions on the template nucleic acid and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers.”

A “barcode” nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” or “constant” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

The terms “identity” and “identical” and their variants, as used herein, when used in reference to two or more sequences, refer to the degree to which the two or more sequences (e.g., nucleotide or polypeptide sequences) are the same. In the context of two or more sequences, the percent identity or homology of the sequences or subsequences thereof indicates the percentage of all monomeric units (e.g., nucleotides or amino acids) that are the same at a given position or region of the sequence (i.e., about 70% identity, preferably 75%, 80%, 85%, 90%, 95%, 97%, 98% or 99% identity). The percent identity canbe over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refer to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U’ denotes uridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. In some embodiments, a nucleotide may comprise any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some, or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label.” In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The phrase “cleavable primers” used herein refers to primers that introduce a restriction endonuclease cleavage site in misprimed nucleic acid amplicons. For example, cleavable primers can be primers for cDNA that introduce a restriction endonuclease cleavage site in misprimed DNA (e.g., genomic DNA or DNA of an antibody conjugated oligonucleotide). As another example, cleavable primers can be primers for genomic DNA that introduce a restriction endonuclease cleavage site in misprimed cDNA. In some embodiments, cleavable primers are designed to amplify cDNA. In some embodiments, cleavable primers are designed to amplify genomic DNA. In some embodiments, cleavable primers are gene specific primers. In some embodiments, cleavable primers are random primers.

Overview

Described herein are embodiments for an improved single-cell analysis workflow that reduces and/or eliminates the presence of primer byproducts and misprimed nucleic acids. Generally, undesired primer byproducts or misprimed nucleic acids are problematic as they result in erroneous sequence reads and/or inaccurate characterization of individual cells. In various embodiments, primer byproducts and misprimed nucleic acids are reduced by implementing cleavable primers. Thus, cleavable primers involved in primer byproducts or misprimed nucleic acids are eliminated such that primer byproducts and misprimed nucleic acids are removed from the subsequent sequencing analysis. In particular embodiments, the cleavable primers participate in the amplification of target nucleic acids. Altogether, the implementation of cleavable primers followed by elimination of primer byproducts and misprimed amplicons enables improved sequence read metrics (e.g., improved percentage of reads after trimming, improved percentage of mapped reads, and/or improved percentage of reads with a valid cell barcode).

Reference is made to FIG. 1A, which depicts an overall system environment including a single cell workflow device 100 and a computational device 180 for conducting single-cell analysis, in accordance with an embodiment. A population of cells 102 are obtained. In various embodiments, the cells 102 can be isolated from a test sample obtained from a subject or a patient. In various embodiments, the cells 102 are healthy cells taken from a healthy subject. In various embodiments, the cells 102 include diseased cells taken from a subject. In one embodiment, the cells 102 include cancer cells taken from a subject previously diagnosed with cancer. For example, cancer cells can be tumor cells available in the bloodstream of the subject diagnosed with cancer. As another example, cancer cells can be cells obtained through a tumor biopsy. Thus, single-cell analysis of the tumor cells enables characterization of cells of the subject's cancer. In various embodiments, the test sample is obtained from a subject following treatment of the subject (e.g., following a therapy such as cancer therapy). Thus, single-cell analysis of the cells enables characterization of cells representing the subject's response to a therapy.

In various embodiments, step 104 is optional and is not performed, as indicated by the dotted lines. In such embodiments, the cells 102 are provided to the single cell workflow device 100 without the antibody-conjugated oligonucleotides. In embodiments where step 104 is performed, the cells 102 are incubated with antibodies. Thus, in various embodiments, an antibody exhibits binding affinity to a target analyte of the cell. For example, an antibody can exhibit binding affinity to a target epitope of a target protein.

In various embodiments, the number of cells incubated with antibodies can be 10² cells, 10³ cells, 10⁴ cells, 10⁵ cells, 10⁶ cells, or 10⁷ cells. In various embodiments, between 10³ cells and 10⁷ cells are incubated with antibodies. In various embodiments, between 10⁴ cells and 10⁶ cells are incubated with antibodies. In various embodiments, varying concentrations of antibodies are incubated with cells. In various embodiments, for an antibody in the protein panel, a concentration of 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM of the antibody is incubated with cells.

In various embodiments, cells 102 are incubated with a plurality of different antibodies. In one embodiment, amongst the plurality of different antibodies, each antibody exhibits binding affinity for an analyte of a panel. For example, each antibody exhibits binding affinity for a protein of a panel. Examples of proteins included in protein panels are described herein. The incubation of cells with antibodies leads to the binding of the antibodies against target epitopes. In various embodiments, a concentration of 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM for each antibody of the antibody panel is incubated with cells.

Following incubation, the cells 102 are washed (e.g., with wash buffer) to remove excess antibodies that are unbound.

In various embodiments, the antibodies are labeled with one or more oligonucleotides, also referred to as antibody oligonucleotides. Such oligonucleotides can be read out with microfluidic barcoding and DNA sequencing, thereby enabling the detection of cell analytes of interest. When an antibody binds its target, the antibody oligonucleotide is carried with it and thus allows the presence of the target analyte to be inferred based on the presence of the oligonucleotide tag. In some implementations, analyzing antibody oligonucleotides provides an estimate of the different epitopes present in the cell.

The single cell workflow device 100 refers to a device that processes individual cells to generate nucleic acids for sequencing. In various embodiments, the single cell workflow device 100 can encapsulate individual cells into emulsions, lyse cells within the emulsions, perform cell barcoding of cell lysate in a second emulsion, and perform a nucleic amplification reaction in the second emulsion. Thus, amplified nucleic acids can be collected and sequenced. In various embodiments, the single cell workflow device 100 includes at least a microfluidic device that is configured to encapsulate cells with reagents to generate cell lysates comprising RNA and/or gDNA, encapsulate cell lysates with reaction mixtures, and perform nucleic acid amplification reactions. For example, the microfluidic device can include one or more fluidic channels that are fluidically connected. Therefore, the combining of an aqueous fluid through a first channel and a carrier fluid through a second channel results in the generation of emulsion droplets. In various embodiments, the fluidic channels of the microfluidic device may have at least one cross-sectional dimension on the order of a millimeter or smaller (e.g., less than or equal to about 1 millimeter). Additional details of microchannel design and dimensions is described in International Patent Application No. PCT/US2016/016444 and U.S. patent application Ser. No. 14/420,646, each of which is hereby incorporated by reference in its entirety. An example of a microfluidic device is the Tapestri™ Platform.

In various embodiments, the single cell workflow device 100 may also include one or more of: (a) a temperature control module for controlling the temperature of one or more portions of the subject devices and/or droplets therein and which is operably connected to the microfluidic device(s), (b) a detection means, i.e., a detector, e.g., an optical imager, operably connected to the microfluidic device(s), (c) an incubator, e.g., a cell incubator, operably connected to the microfluidic device(s), and (d) a sequencer operably connected to the microfluidic device(s). The one or more temperature and/or pressure control modules provide control over the temperature and/or pressure of a carrier fluid in one or more flow channels of a device. As an example, a temperature control module may be one or more thermal cycler that regulates the temperature for performing nucleic acid amplification. The one or more detection means i.e., a detector, e.g., an optical imager, are configured for detecting the presence of one or more droplets, or one or more characteristics thereof, including their composition. In some embodiments, detection means are configured to recognize one or more components of one or more droplets, in one or more flow channel. The sequencer is a hardware device configured to perform sequencing, such as next generation sequencing. Examples of sequencers include Illumina sequencers (e.g., MiniSeg™, MiSeg™, NextSeg™ 550 Series, or NextSeg™ 2000), Roche sequencing system 454, and Thermo Fisher Scientific sequencers (e.g., Ion GeneStudio S5 system, Ion Torrent Genexus System).

The computing device 180 is configured to receive the sequenced reads from the single cell workflow device 100. In various embodiments, the computing device 180 is communicatively coupled to the single cell workflow device 100 and therefore, directly receives the sequence reads from the single cell workflow device 100. The computing device 180 can analyze the sequenced reads to characterize the cells 102. For example, the computing device 180 can construct libraries (e.g., RNA, DNA, or protein libraries) that identify presence or absence of mutations in the cells and/or expression of particular proteins by the cells.

Reference is now made to FIG. 1B, which depicts an embodiment of processing single cells to generate amplified nucleic acid molecules for sequencing. Here, the processing of single cells can be performed by a single cell workflow device (e.g., the single cell workflow device 100 disclosed in FIG. 1A). Specifically, FIG. 1B depicts a workflow process including the steps of cell encapsulation 160, analyte release 165, cell barcoding 170, and target amplification 175 of target nucleic acid molecules.

Generally, the cell encapsulation step 160 involves encapsulating a single cell 102 with reagents 120 into a droplet. In various embodiments, the droplet is formed by partitioning aqueous fluid containing the cell 102 and reagents 120 into a carrier fluid (e.g., oil 115), thereby resulting in a aqueous fluid-in-oil emulsion. The droplet includes encapsulated cell 125 and the reagents 120. The encapsulated cell undergoes an analyte release at step 165. Generally, the reagents cause the cell to lyse, thereby generating a cell lysate 130 within the droplet. The cell lysate 130 includes the contents of the cell, which can include one or more different types of analytes (e.g., RNA transcripts, DNA, protein, lipids, or carbohydrates). In various embodiments, the different analytes of the cell lysate 130 can interact with reagents 120 within the droplet. For example, in particular embodiments, reverse transcriptase in the reagents 120 can reverse transcribe cDNA molecules from RNA transcripts that are present in the cell lysate 130.

In particular embodiments, the reagents 120 include primers. In some embodiments, the primers are gene specific primers. In various embodiments, the primers are reverse primers that are capable of hybridizing to a portion of a nucleic acid, such as a RNA transcript. In such embodiments, the primers enables the reverse transcription of RNA transcripts to generate cDNA. In particular embodiments, the primers are cleavable primers. Further details on cleavable primers is described below.

The cell barcoding step 170 involves encapsulating the cell lysate 130 into a second droplet along with a barcode 145 and/or reaction mixture 140. In various embodiments, the second emulsion is formed by partitioning aqueous fluid containing the cell lysate 130 into immiscible oil 135. As shown in FIG. 1B, the reaction mixture 140 and barcode 145 can be introduced through a separate stream of aqueous fluid, thereby partitioning the reaction mixture 140 and barcode 145 into the second droplet along with the cell lysate 130.

Generally, the reaction mixture 140 enables the performance of a reaction, such as a nucleic acid amplification reaction. In various embodiments, the reaction mixture 140 includes one or more restriction endonucleases capable of cleaving misprimed amplicons such that the nucleic acid sequencing can proceed with improved detection. In such embodiments where the reaction mixture 140 includes one or more restriction endonucleases capable of cleaving misprimed amplicons, the restriction endonucleases cleave the misprimed amplicons here in this droplet at step 170. In various embodiments, the restriction endonucleases cleave the misprimed amplicons following nucleic acid amplification.

The target amplification step 175 involves amplifying target nucleic acids. For example, target nucleic acids of the cell lysate undergo amplification using the reaction mixture 140 in the second emulsion, thereby generating amplicons derived from the target nucleic acids.

Generally, a barcode 145 can label a target nucleic acid to be analyzed (e.g., an analyte of the cell lysate such as genomic DNA, DNA of an antibody conjugated oligonucleotide, or cDNA that has been reverse transcribed from RNA), which enables subsequent identification of the origin of a sequence read that is derived from the target nucleic acid. In various embodiments, multiple barcodes 145 can label multiple target nucleic acid of the cell lysate, thereby enabling the subsequent identification of the origin of large quantities of sequence reads.

As referred herein, the workflow process shown in FIG. 1B is a two-step workflow process in which analyte release 165 from the cell occurs separate from the steps of cell barcoding 170 and target amplification 175. Specifically, analyte release 165 from a cell occurs within a first droplet followed by cell barcoding 170 and target amplification 175 in a second emulsion. In various embodiments, alternative workflow processes (e.g., workflow processes other than the two-step workflow process shown in FIG. 1A) can be employed. For example, the cell 102, reagents 120, reaction mixture 140, and barcode 145 can be encapsulated in a single emulsion. Thus, analyte release 165 can occur within the droplet, followed by cell barcoding 170 and target amplification 175 within the same droplet. Additionally, although FIG. 1B depicts cell barcoding 170 and target amplification 175 as two separate steps, in various embodiments, the target nucleic acid is labeled with a barcode 145 through the nucleic acid amplification step.

FIG. 2 is a flow process for analyzing nucleic acid sequences derived from analytes of the single cell, in accordance with an embodiment. Specifically, FIG. 2 depicts the steps of pooling amplified nucleic acids at step 205, sequencing the amplified nucleic acids at step 210, read alignment at step 215, and characterization at step 220. Generally, the flow process shown in FIG. 2 is a continuation of the workflow process shown in FIG. 1B.

For example, after target amplification at step 175 of FIG. 1B, the amplified nucleic acids 250A, 250B, and 250C are pooled at step 205 shown in FIG. 2. For example, individual droplets containing amplified nucleic acids are pooled and collected, and the immiscible oil of the emulsions is removed. Thus, amplified nucleic acids from multiple cells can be pooled together. FIG. 2 depicts three amplified nucleic acids 250A, 250B, and 250C. In various embodiments, pooled nucleic acids can include hundreds, thousands, or millions of nucleic acids derived from analytes of multiple cells.

In various embodiments, one or more restriction endonucleases are added to the pooled amplified nucleic acids 205. Here, the amplified nucleic acids are held in bulk solution. One or more restriction endonucleases can be added into the bulk solution to cleave misprimed amplicons, thereby removing the barcode from associating with a misprimed amplicon.

In various embodiments, each amplified nucleic acid 250 includes at least a sequence of a target nucleic acid 240 and a barcode 230. In various embodiments, an amplified nucleic acid 250 can include additional sequences, such as any of a universal primer sequence, a random primer sequence, a gene specific primer forward sequence, a gene specific primer reverse sequence, a constant region, or sequencing adapters.

In various embodiments, the amplified nucleic acids 250A, 250B, and 250C are derived from the same single cell and therefore, the barcodes 230A, 230B, and 230C are the same. Therefore, sequencing of the barcodes 230 enables the determination that the amplified nucleic acids 250 are derived from the same cell. In various embodiments, the amplified nucleic acids 250A, 250B, and 250C are pooled and derived from different cells. Therefore, the barcodes 230A, 230B, and 230C are different from one another and sequencing of the barcodes 230 enables the determination that the amplified nucleic acids 250 are derived from different cells.

At step 210, the pooled amplified nucleic acids 250 undergo sequencing to generate sequence reads. For each of one or more amplicons, the sequence read includes at least the sequence of the barcode and the target nucleic acid. Sequence reads originating from individual cells are clustered according to the barcode sequences included in the amplicons. At step 215, the sequence reads for each single cell are aligned (e.g., to a reference genome). Aligning the sequence reads to the reference genome enables the determination of where in the genome the sequence read is derived from. For example, multiple sequence reads generated from amplicons derived from a RNA transcript molecule, when aligned to a position of the genome, can reveal that a gene at the position of the genome was transcribed. As another example, multiple sequence reads generated amplicons derived from a genomic DNA molecule, when aligned to a position of the genome, can reveal the sequence of the gene at the position of the genome.

The alignment of sequence reads at step 215 generates libraries, such as single cell DNA libraries or single cell RNA libraries. Therefore, at step 220, characterization of the libraries and/or the single cells can be performed. In various embodiments, characterization of a library (e.g., DNA library, RNA library, protein library) can involve determining sequencing metrics including, but not limited to: percentage of reads after trimming, percentage of reads with a particular forward primer, percentage of mapped reads, percentage of reads with a valid cell barcode, percentage of exon reads, percentage of intron reads, percentage of mitochondrial reads, and percentage or rRNA reads. In various embodiments, characterization of single cells can involve identifying one or more mutations (e.g., allelic variants, point mutations, single nucleotide variations/polymorphisms, translocations, DNA/RNA fusions, loss of heterozygosity) that are present in one or more of the single cells. Further description regarding characterization of single cells is described in PCT/US2020/026480 and PCT/US2020/026482, each of which is hereby incoiporated by reference in its entirety.

Methods for Performing Single-Cell Analysis

Encapuslation, Analyte Release, Barcoding, and Amplification

Embodiments described herein involve encapsulating one or more cells (e.g., at step 160 in FIG. 1B) to perform single-cell analysis on the one or more cells. In various embodiments, the one or more cells can be isolated from a test sample obtained from a subject or a patient. In various embodiments, the one or more cells are healthy cells taken from a healthy subject. In various embodiments, the one or more cells include cancer cells taken from a subject previously diagnosed with cancer. For example, such cancer cells can be tumor cells available in the bloodstream of the subject diagnosed with cancer. Thus, single-cell analysis of the tumor cells enables cellular and sub-cellular prediction of the subject's cancer. In various embodiments, the test sample is obtained from a subject following treatment of the subject (e.g., following a therapy such as cancer therapy). Thus, single-cell analysis of the cells enables cellular and sub-cellular prediction of the subject's response to a therapy.

In various embodiments, encapsulating a cell with reagents is accomplished by combining an aqueous phase including the cell and reagents with an immiscible oil phase. In one embodiment, an aqueous phase including the cell and reagents are flowed together with a flowing immiscible oil phase such that water in oil emulsions are formed, where at least one emulsion includes a single cell and the reagents. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, the aqueous phase including the cell and reagents need not be simultaneously flowing with the immiscible oil phase. For example, the aqueous phase can be flowed to contact a stationary reservoir of the immiscible oil phase, thereby enabling the budding of water in oil emulsions within the stationary oil reservoir.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell and reagents within an emulsion can then be flowed through the microfluidic device to undergo cell lysis.

Further example embodiments of adding reagents and cells to emulsions can include merging emulsions that separately contain the cells and reagents or picoinjecting reagents into an emulsion. Further description of example embodiments is described in U.S. application Ser. No. 14/420,646, which is hereby incorporated by reference in its entirety.

Generally, the encapsulated cell in an emulsion is lysed to generate cell lysate. In various embodiments, the cell is lysed due to the reagents which include one or more lysing agents that cause the cell to lyse. Examples of lysing agents include detergents such as Triton X-100, NP-40 (e.g., Tergitol-type NP-40 or nonyl phenoxypolyethoxylethanol), as well as cytotoxins. Examples of NP-40 include Thermo Scientific NP-40 Surfact-Amps Detergent solution and Sigma Aldrich NP-40 (TERGITOL Type NP-40). In some embodiments, cell lysis may also, or instead, rely on techniques that do not involve a lysing agent in the reagent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient means of effecting cell lysis may be employed in the methods described herein.

In various embodiments, the reagents include reverse transcriptase which reverse transcribes mRNA transcripts released from the cell to generate corresponding cDNA and further include primers that hybridize with mRNA transcripts, thereby enabling the reverse transcription reaction to occur.

FIGS. 3A-3C depict the processing and releasing of analytes of a single cell in a droplet, in accordance with an embodiment. In FIG. 3A, the cell is lysed, as indicated by the dotted line of the cell membrane. In some embodiments, the reagents include a detergent, such as NP40 (e.g., 0.01% or 1.0% NP40) or Triton-X100, which causes the cell to lyse. The lysed cell includes analytes such as RNA transcripts within the cytoplasm of the cell as well as packaged DNA 302, which refers to the organization of DNA with histones, thereby forming nucleosomes that are packaged as chromatin. Additionally, the emulsion 300A includes antibody oligonucleotides 305. As shown in FIG. 3A, the reagents included in the emulsion 300A further includes reverse transcriptase (abbreviated as “RT” 310). Furthermore, the reagents included in the emulsion 300A further includes an enzyme 312 that digests the packaged DNA 302. In various embodiments, the enzyme 312 is proteinase K.

FIG. 3B depicts the emulsion 300B in a second state as reverse transcriptase performs reverse transcription on the RNA transcripts and the enzymes 312 digest the packaged DNA 302. Here, reverse transcription occurs through the use of reverse transcription primers that hybridize with a region of the RNA transcript. Thus, reverse transcriptase generates a complementary cDNA strand off of the RNA transcript starting at the hybridized primer.

FIG. 3C depicts the emulsion 300C in a third state that includes synthesized cDNA 306. Emulsion 300C also includes freed gDNA 340 that is released from the packaged DNA 302. Furthermore, emulsion 300C includes the antibody oligonucleotides 305.

In various embodiments, the emulsion 300C can be exposed to conditions to inactivate the enzymes 312. In various embodiments, the emulsion 300C is exposed to an elevated temperature of at least 50° C. to inactivate the enzymes 312. In various embodiments, the emulsion 300C is exposed to an elevated temperature of at least 60° C. to inactivate the enzymes 312. In various embodiments, the emulsion 300C is exposed to an elevated temperature of at least 70° C. to inactivate the enzymes 312. In various embodiments, the emulsion 300C is exposed to an elevated temperature of at least 80° C. to inactivate the enzymes 312.

Returning to the step of cell barcoding 170 in FIG. 1B, it includes encapsulating a cell lysate 130 with a reaction mixture 140 and a barcode 145. Generally, the reaction mixture includes reactants sufficient for performing a reaction, such as nucleic acid amplification, on analytes of the cell lysate. In various embodiments, the reaction mixture 140 includes components, such as primers, for performing the nucleic acid reaction on the analytes. Such primers are capable of acting as a point of initiation of synthesis along a complementary strand when placed under conditions in which synthesis of a primer extension product which is complementary to a nucleic acid strand is catalyzed. In various embodiments, the primers for performing the nucleic acid reaction are cleavable primers.

In various embodiments, a cell lysate is encapsulated with a reaction mixture and a barcode by combining an aqueous phase including the reaction mixture and the barcode with the cell lysate and an immiscible oil phase. In one embodiment, an aqueous phase including the reaction mixture and the barcode are flowed together with a flowing cell lysate and a flowing immiscible oil phase such that water in oil emulsions are formed, where at least one emulsion includes a cell lysate, the reaction mixture, and the barcode. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell lysate, reaction mixture, and barcode within an emulsion can then be flowed through the microfluidic device to perform amplification of target nucleic acids.

Further example embodiments of adding reaction mixture and barcodes to emulsions can include merging emulsions that separately contain the cell lysate and reaction mixture and barcodes or picoinjecting the reaction mixture and/or barcode into an emulsion. Further description of example embodiments of merging emulsions or picoinjecting substances into an emulsion is found in U.S. application Ser. No. 14/420,646, which is hereby incorporated by reference in its entirety.

In various embodiments, subsequent to adding the reaction mixture and barcode to an emulsion, the misprimed nucleic acid amplicons are cleaved. Misprimed nucleic acid amplicons are cleaved to remove their subsequent detection by sequencing. In various embodiments, the cleavage of misprimed nucleic acid amplicons reduces or eliminates the presence of the misprimed nucleic acid amplicons. This can include products of cleavable primers that have formed primer byproducts and misprimed cleavable primers (e.g., cDNA primers that have primed a different nucleic acid such as genomic DNA or DNA of antibody conjugated oligonucleotides).

The emulsion may be incubated under conditions that facilitates the nucleic acid amplification reaction. In various embodiments, the emulsion may be incubated on the same microfluidic device as was used to add the reaction mixture and/or barcode, or may be incubated on a separate device. In certain embodiments, incubating the emulsion under conditions that facilitates nucleic acid amplification is performed on the same microfluidic device used to encapsulate the cells and lyse the cells. Incubating the emulsions may take a variety of forms. In certain aspects, the emulsions containing the reaction mix, barcode, and cell lysate may be flowed through a channel that incubates the emulsions under conditions effective for nucleic acid amplification. Flowing the microdroplets through a channel may involve a channel that snakes over various temperature zones maintained at temperatures effective for PCR. Such channels may, for example, cycle over two or more temperature zones, wherein at least one zone is maintained at about 65° C. and at least one zone is maintained at about 95° C. As the drops move through such zones, their temperature cycles, as needed for nucleic acid amplification. The number of zones, and the respective temperature of each zone, may be readily determined by those of skill in the art to achieve the desired nucleic acid amplification. Additionally, the extent of nucleic amplification can be controlled by modulating the concentration of the reactants in the reaction mixture. In some instances, this is useful for fine tuning of the reactions in which the amplified products are used.

In various embodiments, following nucleic acid amplification, restriction endonucleases are added to the emulsion at appropriate concentrations for cleavage. In various embodiments, the emulsion is then incubated under conditions that facilitate cleavage of the misprimed nucleic acid amplicons by the restriction nucleases.

In various embodiments, following nucleic acid amplification, emulsions containing the amplified nucleic acids are collected. In various embodiments, the emulsions are collected in a well, such as a well of a microfluidic device. In various embodiments, the emulsions are collected in a reservoir or a tube, such as an Eppendorf tube. Once collected, the amplified nucleic acids across the different emulsions are pooled. In one embodiment, the emulsions are broken by providing an external stimuli to pool the amplified nucleic acids. In one embodiment, the emulsions naturally aggregate over time given the density differences between the aqueous phase and immiscible oil phase. Thus, the amplified nucleic acids pool in the aqueous phase.

In various embodiments, restriction endonucleases are added to the pooled amplified nucleic acids and therefore, cleavage of misprimed amplicons can occur in bulk (e.g., outside of emulsions). In such embodiments, restriction endonucleases are not previously added (e.g., not previously added into the emulsions). In various embodiments, the solution containing the pooled amplicons and restriction endonucleases can be incubated under conditions that facilitate cleavage of the misprimed nucleic acid amplicons by the restriction endonucleases.

Following pooling, the amplified nucleic acids can undergo further preparation for sequencing. For example, sequencing adapters can be added to the pooled nucleic acids. Example sequencing adapters are P5 and P7 sequencing adapters. The sequencing adapters enable the subsequent sequencing of the nucleic acids.

Sequencing and Read Alignment

Amplified nucleic acids are sequenced to obtain sequence reads for generating a sequencing library. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing, libraries of NGS fragments are cloned in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing is described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell. These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform is found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′ end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology is found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope is found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serve as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′ phosphosulfate. Luciferase uses ATP to convert luciferin to oxyluciferin, and as a result of this reaction, light is generated that is detected and analyzed. Additional details for performing sequencing 454 is found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology is found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, sequencing reads obtained from the NGS methods can be filtered by quality and grouped by barcode sequence using any algorithms known in the art, e.g., Python script barcodeCleanup.py . In some embodiments, a given sequencing read may be discarded if more than about 20% of its bases have a quality score (Q-score) less than Q20, indicating a base call accuracy of about 99%. In some embodiments, a given sequencing read may be discarded if more than about 5%, about 10%, about 15%, about 20%, about 25%, about 30% have a Q-score less than Q10, Q20, Q30, Q40, Q50, Q60, or more, indicating a base call accuracy of about 90%, about 99%, about 99.9%, about 99.99%, about 99.999%, about 99.9999%, or more, respectively.

In some embodiments, all sequencing reads associated with a barcode containing less than 50 reads may be discarded to ensure that all barcode groups, representing single cells, contain a sufficient number of high-quality reads. In some embodiments, all sequencing reads associated with a barcode containing less than 30, less than 40, less than 50, less than 60, less than 70, less than 80, less than 90, less than 100 or more may be discarded to ensure the quality of the barcode groups representing single cells.

Of note, misprimed amplicons that have undergone cleavage (e.g., by a restriction endonuclease) no longer have a barcode sequence attached to the misprimed amplicon. Therefore, although the sequences of the misprimed amplicons may be sequenced, they are not associated with a barcode sequence and therefore, are not assigned to a particular cell because of the lack of a barcode sequence.

Sequence reads with common barcode sequences (e.g., meaning that sequence reads originated from the same cell) may be aligned to a reference genome using known methods in the art to determine alignment position information. The alignment position information may indicate a beginning position and an end position of a region in the reference genome that corresponds to a beginning nucleotide base and end nucleotide base of a given sequence read. A region in the reference genome may be associated with a target gene or a segment of a gene. Example aligner algorithms include BWA, Bowtie, Spliced Transcripts Alignment to a Reference (STAR), Tophat, or HISAT2. Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis.

In various embodiments, sequencing and read alignment results in generation of a nucleic acid library (e.g., a RNA library and/or a DNA library). In various embodiments, nucleic acid libraries can be evaluated based on one or more sequence read metrics. Example sequence read metrics include percentage of reads after trimming, percentage of reads with the forward primer, percentage of mapped reads, and percentage of reads with a valid cell barcode. Generally, the single cell analysis workflow disclosed herein involving the implementation of cleavable primers followed by cleavage of the misprimed amplicons including the cleavable primers enables improved sequence read metrics in comparison to a single cell analysis workflow that does not implement cleavable primers.

In various embodiments, the single cell analysis workflow disclosed herein involving the implementation of cleavable primers followed by cleavage of the misprimed nucleic acid amplicons achieves at least a 2-fold increase in percentage of mapped reads in comparison to a workflow process that implements non-cleavable primers. In various embodiments, the single cell analysis workflow disclosed herein involving the implementation of cleavable primers followed by cleavage of the misprimed amplicons achieves at least a 3-fold increase, at least a 4-fold increase, or at least a 5-fold increase in percentage of mapped reads in comparison to a workflow process that implements non-cleavable primers.

In various embodiments, the single cell analysis workflow disclosed herein involving the implementation of cleavable primers followed by cleavage of the misprimed nucleic acid amplicons achieves at least a 1.2-fold increase in percentage of reads after trimming in comparison to a workflow process that implements oligo dT primers as opposed to cleavable primers. In various embodiments, the single cell analysis workflow disclosed herein involving the implementation of cleavable primers followed by cleavage of the misprimed nucleic acid amplicons achieves at least a 2-fold increase, at least a 3-fold increase, at least a 4-fold increase, or at least a 5-fold increase in percentage of reads after trimming in comparison to a workflow process that implements non-cleavable primers.

Example Implementation of Cleavable Primers for Removing Misprimed Amplicons

Described herein are implementations of cleavable primers for removing misprimed amplicons, including misprimed DNA amplicons and/or misprimed cDNA amplicons. The sections below describe the processes of barcoding and amplifying antibody-conjugated oligonucleotides (e.g., FIG. 4A), barcoding and amplifying genomic DNA (e.g., FIG. 4B), and barcoding and amplifying cDNA derived from RNA transcripts (e.g., FIGS. 4C and 4D). Although these embodiments are described independent from one another, in a mulit-omic analysis that involves analyzing protein expression, DNA, and RNA, parts of the processes shown in FIGS. 4A-4D can occur simultaneously. Thus, this can lead to mispriming amplicons. In various embodiments, these misprimed amplicons include cleavable primers and therefore, the misprimed amplicons can be cleaved and eliminated.

Example Barcoding of Antibody-Conjugated Oligonucleotide

FIG. 4A illustrates the priming and barcoding of an antibody-conjugated oligonucleotide, in accordance with an embodiment. Specifically, FIG. 4A depicts step 410 involving the priming of the antibody oligonucleotide 304 and further depicts step 420 which involves the barcoding and amplification of the antibody oligonucleotide 304. In various embodiments, step 410 occurs within a first emulsion during which cell lysis occurs (e.g., cell encapsulation 160 and analyte release 165 in FIG. 1B) and step 420 occurs within a second emulsion (e.g., cell barcoding 170 and nucleic acid amplification 175 in FIG. 1B). In such embodiments, the primer 405 is provided in the reagents and the bead barcode is provided with the reaction mixture. In some embodiments, both steps 410 and 420 occur within a single emulsion (e.g., a second emulsion). In such embodiments, the primer 405 and the bead barcode shown in FIG. 4A are provided with the reaction mixture.

The antibody oligonucleotide 305 is conjugated to an antibody. In various embodiments, an antibody oligonucleotide 305 includes a PCR handle, a tag sequence (e.g., an antibody tag), and a capture sequence that links the oligonucleotide to the antibody. In various embodiments, the antibody oligonucleotide 305 is conjugated to a region of the antibody, such that the antibody's ability to bind a target epitope is unaffected. For example, the antibody oligonucleotide 305 can be linked to a Fc region of the antibody, thereby leaving the variable regions of the antibody unaffected and available for epitope binding. In various the antibody oligonucleotide 305 can include a unique molecular identifier (UMI). In various embodiments, the UMI can be inserted before or after the antibody tag. In various embodiments, the UMI can flank either end of the antibody tag. In various embodiments, the UMI enables the identification of the particular antibody oligonucleotide 305 and antibody combination.

In various embodiments, the antibody oligonucleotide 305 includes more than one PCR handle. For example, the antibody oligonucleotide 305 can include two PCR handles, one on each end of the antibody oligonucleotide 305. In various embodiments, one of the PCR handles of the antibody oligonucleotide 305 is conjugated to the antibody. Here, forward and reverse primers can be provided that hybridize with the two PCR handles, thereby enabling amplification of the antibody oligonucleotide 305.

Generally, the antibody tag of the antibody oligonucleotide 305 enables the subsequent identification of the antibody (and corresponding protein). For example, the antibody tag can serve as an identifier e.g., a barcode for identifying the type of protein for which the antibody binds to. In various embodiments, antibodies that bind to the same target are each linked to the same antibody tag. For example antibodies that bind to the same epitope of a target protein are each linked to the same antibody tag, thereby enabling the subsequent determination of the presence of the target protein. In various embodiments, antibodies that bind different epitopes of the same target protein can be linked to the same antibody tag, thereby enabling the subsequent determination of the presence of the target protein.

In some embodiments, an oligonucleotide sequence is encoded by its nucleobase sequence and thus confers a combinatorial tag space far exceeding what is possible with conventional approaches using fluorescence. For example, a modest tag length of ten bases provides over a million unique sequences, sufficient to label an antibody against every epitope in the human proteome. Indeed, with this approach, the limit to multiplexing is not the availability of unique tag sequences but, rather, that of specific antibodies that can detect the epitopes of interest in a multiplexed reaction.

Step 410 depicts the priming of the antibody oligonucleotide 305 by a primer 405. Here, the PCR handle of the primer 405 is complementary to the PCR handle of the antibody oligonucleotide 305. Thus, the primer 405 primes the antibody oligonucleotide 305 given the hybridization of the PCR handles. In various embodiments, extension occurs from the PCR handle of the antibody oligonucleotide 304 (as indicated by the dotted arrow). In various embodiments, extension occurs from the PCR handle of the primer 405, thereby generating a nucleic acid with the antibody tag and capture sequence.

Step 420 depicts the barcoding of the antibody oligonucleotide 304. As shown in FIG. 4A, the barcode (e.g., cell barcode) is releasably attached to a bead and is further linked to a common sequence. Here, the common sequence linked to the cell barcode is complementary to the common sequence linked to the PCR handle, antibody tag, and capture sequence. The antibody oligonucleotide is extended to include the common sequence and cell barcode.

In various embodiments, the antibody oligonucleotide is amplified, thereby generating amplicons with the cell barcode, common sequence, PCR handle, antibody tag, and capture sequence. In various embodiments, during amplification, mispriming of the antibody oligonucleotide amplicons can occur. For example, amplicons can be misprimed by a cleavable primer (e.g., a cleavable primer that should have primed a cDNA sequence). Thus, in such scenarios, the misprimed antibody oligonucleotide amplicons can be cleaved, as is described in further detail below.

Example Implementation of Cleavable Primers for DNA Sequencing

Embodiments disclosed herein refer to a single cell workflow process for targeted DNA sequencing using cleavable primers. FIG. 4B depicts the amplification and barcoding of nucleic acids derived from gDNA, in accordance with an embodiment. In various embodiments, the process shown in FIG. 4B is performed in a second droplet (e.g., during cell barcoding 170 in FIG. 1B). In various embodiments, the genomic DNA 455 can be the freed gDNA 340 shown in FIG. 3C, that has been released from its packaging.

A primer 465, such as a reverse primer, which is added in the reaction mixture, hybridizes with a region of the genomic DNA 455. As shown in FIG. 4B, the primer 465 can further include a PCR handle, which can be a constant region.

In the middle panel of FIG. 4B, a forward primer 475, which is also added in the reaction mixture, hybridizes with a region of the genomic DNA 455. In various embodiments, the forward primer 475 is a cleavable primer. Therefore, the forward primer 475 includes a cleavage site 470. In some embodiments, the forward primer 475 does not include a cleavable primer. For example, in a scenario in which the workflow includes a cleavable primer that targets a cDNA molecule, the forward primer 475 here is not a cleavable primer. As shown in FIG. 4B, the forward primer 475 may also include a PCR handle, which can be a constant region. Thus, the PCR handle can hybridize with another PCR handle of a cell barcode (shown as “Cell BC” in FIG. 4B). Thus, over subsequent nucleic acid amplification cycles, the forward primer (including the cleavage site 470) as well as the cell barcode are incorporated into the genomic DNA amplicon 472.

Example Implementation of Cleavable Primers for Whole Transcriptome Sequencing

Embodiments disclosed herein refer to a single cell workflow process for whole transcriptome sequencing using cleavable primers. In various embodiments, the whole transcriptome sequencing workflow uses cleavable primers that introduce a restriction endonuclease cleavage site into misprimed nucleic acid amplicons.

FIGS. 4C and 4D depict the amplification and barcoding of nucleic acids derived from RNA, in accordance with an embodiment. In various embodiments, the process shown in FIG. 4C is performed in a first droplet (e.g., during cell encapsulation 160 and analyte release 165 in FIG. 1B). The process shown in FIG. 4D is performed in a second droplet (e.g., during cell barcoding 170 in FIG. 1B). In various embodiments, the RNA 481 can be the RNA 304 shown in FIG. 3A. Additionally the generated cDNA 483 and cDNA 486 can be cDNA 306 shown in FIG. 3C.

As shown in FIG. 4C, the RNA 481 is primed by a reverse transcription (RT) primer 482. Here, the RT primer 482 includes a read sequence. This enables reverse transcription to occur, thereby producing a cDNA 483 that is complementary to the RNA 481. Given that the reverse transcription extends from the RT primer 482, the RT primer is included in the cDNA 483 molecule. A random primer 484, which is included in the reagents, hybridizes with a region of the cDNA 483. As shown in FIG. 4C, the random primer 484 can be a cleavable primer. Thus, the random primer 484 includes a cleavage site, which is shown to be located between a constant region (referred to as “FWD site” in FIG. 4C) and the portion of the random primer 484 that hybridizes with the cDNA 483. In some embodiments, the random primer 484 is not a cleavable primer. For example, in a scenario in which the workflow includes a cleavable primer that targets a DNA molecule, the random primer 484 here is not a cleavable primer.

As shown in the bottom panel of FIG. 4C, nucleic acid extension occurs beginning at the random primer 484 to generate a second cDNA strand 486. FIG. 4D shows the second cDNA strand 486, which is primed by a forward primer 490 and a reverse primer 491. Here, the forward primer 490 and the reverse primer 491 can be a primer pair. In various embodiments, although not shown in FIG. 4D, the forward primer 490 can be a cleavable primer. For example, the forward primer 490 can include a cleavage site located between the constant region (labeled as “Const”) and the portion of the forward primer 490 that hybridizes with the FWD site of the random primer. The constant region of the forward primer 490 can hybridize with a region of a cell barcode (labeled as “CBC” 492 in FIG. 4D). Thus, over subsequent nucleic acid amplification cycles, a cDNA amplicon 494 can be generated which incorporates a cleavage site (e.g., cleavage site 485) and the cell barcode 492.

Removing Misprimed DNA Amplicons

Embodiments disclosed herein include the use of a cleavable primer, such as a random primer that includes a cleavage site (e.g., as shown in FIGS. 4C and 4D). During multi-omic analysis, the random primer including the cleavage site may generate primer byproducts and/or misprime amplicons.

FIG. 5A depicts the nucleic acid amplicons arising from RNA primers including primer dimers, misprimed gDNA and misprimed antibody tag DNA. Specifically, FIG. 5A depicts primer byproducts (e.g., primer dimers 501 and 502) as well as misprimed amplicons (e.g., amplicons 503, 504, and 505) when using a cleavable primer (e.g., random primer including a cleavage site) and a non-cleavable primer (e.g., “fwr primer”), which is a gene specific primer designed to target a DNA sequence. As shown in FIG. 5A, one or more of the primer byproducts and misprimed amplicons include a cleavable primer 508. As referred to in FIGS. 5A and 5B, the cleavable primer 508 includes a constant region (referred to as “Seq8F” 507 in FIG. 5A) and further includes a cleavage site 506.

For example, primer dimer 501 includes the cleavable random primer 508 which includes a cleavage site 506 and a constant region (referred to as “Seq8F” 507 in FIG. 5A) and a reverse primer (Rev primer+Rd2) that was intended to prime a DNA molecule. Primer dimer 502 includes a forward primer (Seq8F+Fwr primer) that was intended to prime a DNA and a reverse primer (32092+Rd2) that was intended to prime a cDNA. Misprimed amplicon 503 represents an amplicon with DNA primer crosstalk. Specifically, the nucleic acid is a cDNA molecule derived from a RNA transcript, but it has been misprimed by a forward primer (e.g., Seq8F+Fwr primer) that was intended to prime a DNA molecule. Misprimed amplicon 504 and amplicon 505 represent amplicons with RNA primer crosstalk. Specifically, amplicon 504 includes a gDNA molecule whereas amplicon 505 includes a DNA corresponding to the antibody tag. In each of amplicon 504 and amplicon 505, the nucleic acids have been misprimed by the random primer (e.g., Seq8F, cleavage site 506, and Rdm 9N 508) which was intended to prime a gDNA molecule.

Notably, in each of misprimed amplicon 504 and misprimed amplicon 505, the cleavage site 506 is located between the nucleic acid sequence of interest (e.g., gDNA for 504 and antibody tag for 505) and the barcode sequence (labeled as BC1+Const1+BC2).

FIG. 5B depicts the cleavage of misprimed nucleic acid amplicons arising from RNA primers, in accordance with the embodiment shown in FIG. 5A. Here, the primer dimer 501, the misprimed amplicon 504, and the misprimed amplicon 505 are exposed to a restriction endonuclease 511. The restriction endonuclease cleaves the molecules at the respective cleavage sites 506. Again, the cleavage sites 506 in each of misprimed amplicon 504 and misprimed amplicon are located between the nucleic acid sequence of interest (e.g., gDNA for 504 and antibody tag for 505) and the barcode sequence (labeled as BC1+Const1+BC2). Therefore, the cleavage removes the association between the barcode sequence and the nucleic acid sequence interest.

Removing Misprimed cDNA Amplicons Derived from RNA Transcripts

Embodiments disclosed herein include the use of a cleavable primer, such as a forward primer that includes a cleavage site (e.g., forward primer 475 as shown in FIG. 4B). During multi-omic analysis, the forward primer including the cleavage site may generate primer byproducts and/or misprime amplicons.

FIG. 6A depicts the nucleic acid amplicons arising from DNA primers including primer dimers and misprimed cDNA. Specifically, FIG. 6A depicts primer byproducts (e.g., primer dimers 601 and 602) as well as misprimed amplicons (e.g., amplicons 603, 604, and 605) when using a cleavable primer (e.g., forward primer including a cleavage site labeled as “Fwr primer”) and a non-cleavable primer (e.g., the random primer “Rdm 9N”). As shown in FIG. 6A, one or more of the primer byproducts and misprimed amplicons include a cleavable primer 608. As referred to in FIGS. 6A and 6B, the cleavable primer 608 includes a constant region (referred to as “Seq8F” 607 in FIG. 5A) and further includes a cleavage site 606.

Primer dimer 601 includes a random primer (Seq8F+Rdm 9N in FIG. 6A) and a reverse primer (Rev primer+Rd2) that was intended to prime a DNA molecule. Here, the random primer is not a cleavable primer. Primer dimer 602 includes a cleavable forward primer (Seq8F 607, cleavage site 606, and Fwr primer 608) that was intended to prime a DNA and a reverse primer (32092+Rd2) that was intended to prime a cDNA. Misprimed amplicon 603 represents an amplicon with DNA primer crosstalk. Specifically, the nucleic acid is a cDNA molecule derived from a RNA transcript, but it has been misprimed by the cleavable forward primer (e.g., Seq8F 607, cleavage site 606, and Fwr primer 608) that was intended to prime a DNA molecule. Misprimed amplicon 604 and amplicon 605 represent amplicons with RNA primer crosstalk. Specifically, amplicon 604 includes a gDNA molecule whereas amplicon 605 includes a DNA corresponding to the antibody tag. In each of amplicon 604 and amplicon 605, the nucleic acids have been misprimed by the random primer which was intended to prime a gDNA molecule.

Notably, in the misprimed amplicon 603, the cleavage site 606 is located between the nucleic acid sequence of interest (e.g., cDNA) and the barcode sequence (labeled as BC1+Const1+BC2).

FIG. 6B depicts the cleavage of misprimed nucleic acid amplicons arising from DNA primers, in accordance with the embodiment shown in FIG. 6A. Here, the primer dimer 602 and the misprimed amplicon 603 are exposed to a restriction endonuclease 611. The restriction endonuclease cleaves the molecules at the respective cleavage sites 606. Again, the cleavage site 606 in the misprimed amplicon 603 is located between the nucleic acid sequence of interest (e.g., cDNA) and the barcode sequence (labeled as BC1+Const1+BC2). Therefore, the cleavage removes the association between the barcode sequence and the nucleic acid sequence interest.

Barcodes and Barcoded Beads

Embodiments of the invention involve providing one or more barcode sequences for labeling analytes of a single cell during step 170 shown in FIG. 1B. The one or more barcode sequences are encapsulated in an emulsion with a cell lysate derived from a single cell. As such, the one or more barcodes label analytes of the cell, thereby enabling the subsequent determination that sequence reads derived from the analytes originated from the cell.

In various embodiments, a plurality of barcodes are added to an emulsion with a cell lysate. In various embodiments, the plurality of barcodes added to an emulsion includes at least 10², at least 10³, at least 10⁴, at least 10⁵, at least 10⁵, at least 10⁶, at least 10⁷, or at least 10⁸ barcodes. In various embodiments, the plurality of barcodes added to an emulsion have the same barcode sequence. In various embodiments, the plurality of barcodes added to an emulsion comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a barcode sequence and a UMI are incorporated into a barcode. Generally, a UMI is used to distinguish between molecules of a similar type within a population or group, whereas a barcode sequence is used to distinguish between populations or groups of molecules that are derived from different cells. Thus, a UMI can be used to count or quantify numbers of particular molecules (e.g., quantify number of RNA transcripts). In some embodiments, where both a UMI and a barcode sequence are utilized, the UMI is shorter in sequence length than the barcode sequence. The use of barcodes is further described in U.S. patent application Ser. No. 15/940,850, which is hereby incorporated by reference in its entirety.

In some embodiments, the barcodes are single-stranded barcodes. Single-stranded barcodes can be generated using a number of techniques. For example, they can be generated by obtaining a plurality of DNA barcode molecules in which the sequences of the different molecules are at least partially different. These molecules can then be amplified so as to produce single stranded copies using, for instance, asymmetric PCR. Alternatively, the barcode molecules can be circularized and then subjected to rolling circle amplification. This will yield a product molecule in which the original DNA barcoded is concatenated numerous times as a single long molecule.

In some embodiments, circular barcode DNA containing a barcode sequence flanked by any number of constant sequences can be obtained by circularizing linear DNA. Primers that anneal to any constant sequence can initiate rolling circle amplification by the use of a strand displacing polymerase (such as Phi29 polymerase), generating long linear concatemers of barcode DNA.

In various embodiments, barcodes can be linked to a primer sequence that enables the barcode to label a target nucleic acid. In one embodiment, the barcode is linked to a forward primer sequence. In various embodiments, the forward primer sequence is a gene specific primer that hybridizes with a forward target of a nucleic acid. In various embodiments, the forward primer sequence is a constant region, such as a PCR handle, that hybridizes with a complementary sequence attached to a gene specific primer. The complementary sequence attached to a gene specific primer can be provided in the reaction mixture (e.g., reaction mixture 140 in FIG. 1B). Including a constant forward primer sequence on barcodes may be preferable as the barcodes can have the same forward primer and need not be individually designed to be linked to gene specific forward primers.

In various embodiments, barcodes can releasably attached to a support structure, such as a bead. Therefore, a single bead with multiple copies of barcodes can be partitioned into an emulsion with a cell lysate, thereby enabling labeling of analytes of the cell lysate with the barcodes of the bead. Example beads include solid beads (e.g., silica beads), polymeric beads, or hydrogel beads (e.g., polyacrylamide, agarose, or alginate beads). Beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-split cycle. Additional details of example beads and their synthesis is described in International Application No. PCT/US2016/016444, which is hereby incorporated by reference in its entirety.

Reagents

Embodiments described herein include the encapsulation of a cell with reagents within an emulsion. In various embodiments, the reagents interact with the encapsulated cell under conditions in which the cell is lysed, thereby releasing target analytes of the cell. The reagents can further interact with target analytes to prepare for subsequent barcoding and/or amplification.

In various embodiments, the reagents include one or more lysing agents that cause the cell to lyse. Examples of lysing agents include detergents such as Triton X-100, Nonidet P-40 (NP40) as well as cytotoxins. In various embodiments, the reagents further include agents that interact with target analytes that are released from a single cell. One example of such an agent includes reverse transcriptase which reverse transcribes messenger RNA transcripts released from the cell to generate corresponding cDNA.

In various embodiments, the reagents encapsulated with the cell include ddNTPs, inhibitors such as ribonuclease inhibitor, and stabilization agents such as dithothreitol (DTT). In various embodiments, the reagents further include proteases that assist in the lysing of the cell and/or accessing of genomic DNA. In various embodiments, proteases in the reagents can include any of proteinase K, pepsin, protease—subtilisin Carlsberg, protease type X—Bacillus thermoproteolyticus, or protease type XIII—Aspergillus Saitoi. In various embodiments, the reagents include deoxyribonucleotide triphosphate (dNTP) reagents including deoxyadenosine triphosphate, deoxycytosine triphosphate, deoxyguanine triphosphate, and deoxythymidine triphosphate.

In various embodiments, the reagents include agents that interact with target analytes that are released from a single cell. For example, the reagents include reverse transcriptase which reverse transcribes mRNA transcripts released from the cell to generate corresponding cDNA. As another example, the reagents include primers that hybridize with mRNA transcripts, thereby enabling the reverse transcription reaction to occur.

In various embodiments, the reagents include forward primers for hybridizing with a nucleic acid. In various embodiments, the reagents include a random primer that hybridizes with a nucleic acid, such as a cDNA strand that was reverse transcribed from a RNA transcript. The random primer can be useful for whole transcriptome sequencing. In various embodiments, the forward primer or random primer is a cleavable primer that includes a cleavage site. For example, the forward primer or random primer maybe include a cleavage site within the primer that enables subsequent cleavage of misprimed amplicons that include the cleavable primer.

Reaction Mixture

As described herein, a reaction mixture is provided into an emulsion with a cell lysate (e.g., see cell barcoding step 170 in FIG. 1B). Generally, the reaction mixture includes reactants sufficient for performing a reaction, such as nucleic acid amplification, on analytes of the cell lysate.

In various embodiments, the reaction mixture includes primers that are capable of acting as a point of initiation of synthesis along a complementary strand when placed under conditions in which synthesis of a primer extension product which is complementary to a nucleic acid strand is catalyzed. In various embodiments, the reaction mixture includes the four different deoxyribonucleoside triphosphates (adenosine, guanine, cytosine, and thymine). In various embodiments, the reaction mixture includes enzymes for nucleic acid amplification. Examples of enzymes for nucleic acid amplification include DNA polymerase, thermostable polymerases for thermal cycled amplification, or polymerases for multiple-displacement amplification for isothermal amplification. Other, less common forms of amplification may also be applied, such as amplification using DNA-dependent RNA polymerases to create multiple copies of RNA from the original DNA target which themselves can be converted back into DNA, resulting in, in essence, amplification of the target. Living organisms can also be used to amplify the target by, for example, transforming the targets into the organism which can then be allowed or induced to copy the targets with or without replication of the organisms.

In various embodiments, the reaction mixture includes cleavable primers that participate in the nucleic acid amplification reaction. In various embodiments, the cleavable primer is a gene specific primer. In various embodiments, the cleavable primer is a gene specific primer that hybridizes with a complementary sequence of a genomic DNA molecule, an example of which is shown and described in above in reference to FIG. 4B. In various embodiments, the cleavable primer is a forward primer.

In various embodiments, the reagents include deoxyribonucleotide triphosphate (dNTP) reagents including deoxyadenosine triphosphate, deoxycytosine triphosphate, deoxyguanine triphosphate, and deoxythymidine triphosphate.

The extent of nucleic amplification can be controlled by modulating the concentration of the reactants in the reaction mixture. In some instances, this is useful for fine tuning of the reactions in which the amplified products are used.

Primers

Embodiments of the invention described herein use primers to conduct the single-cell analysis. For example, primers are implemented during the workflow process shown in FIG. 1B. Primers can be used to prime (e.g., hybridize) with specific sequences of nucleic acids of interest, such that the nucleic acids of interest can be processed (e.g., reverse transcribed, barcoded, and/or amplified). Additionally, primers enable the identification of target regions following sequencing.

In various embodiments, primers described herein are between 5 and 50 nucleobases in length. In various embodiments, primers described herein are between 7 and 45 nucleobases in length. In various embodiments, primers described herein are between 10 and 40 nucleobases in length. In various embodiments, primers described herein are between 12 and 35 nucleobases in length. In various embodiments, primers described herein are between 15 and 32 nucleobases in length. In various embodiments, primers described herein are between 18 and 30 nucleobases in length. In various embodiments, primers described herein are between 18 and 25 nucleobases in length.

Referring again to FIG. 1B, in various embodiments, primers can be included in the reagents 120 that are encapsulated with the cell 102. In various embodiments, primers included in the reagents are useful for priming RNA transcripts and enabling reverse transcription of the RNA transcripts. In various embodiments, primers in the reagents 120 can include RNA primers for priming RNA and/or for priming genomic DNA. In various embodiments, the primers included in the reagents are cleavable primers. Cleavable primers introduce a cleavage site into misprimed amplicons, thereby enabling the cleaving of misprimed amplicons such that they are not subsequently identified during sequencing.

In various embodiments, primers can be included in the reaction mixture 140 that is encapsulated with the cell lysate 130. In various embodiments, primers included in the reaction mixture are useful for priming nucleic acids (e.g., cDNA, gDNA, and/or amplicons of cDNA/gDNA) and enabling nucleic acid amplification of the nucleic acids. Such primers in the reaction mixture 140 can include cDNA primers for priming cDNA that have been reverse transcribed from RNA and/or DNA primers for priming genomic DNA and/or for priming products that have been generated from the genomic DNA. In various embodiments, primers of the reagents and primers of the reaction mixture form primer sets (e.g., forward primer and reverse primer) for a region of interest on a nucleic acid. In various embodiments, primers can be included in or linked with a barcode 145 that is encapsulated with the cell lysate 130. Further description and examples of primers that are used in a single-cell analysis workflow process is described in U.S. application Ser. No. 16/749,731, which is hereby incorporated by reference in its entirety.

In various embodiments, the number of primers in any of the reagents, the reaction mixture, or with barcodes may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

For targeted nucleic acid (e.g., targeted DNA or targeted RNA) sequencing, primers in the reagents (e.g., reagents 120 in FIG. 1B) may include primers that are complementary to a target on a nucleic acid of interest (e.g., DNA or RNA). In various embodiments, primers in the reagents are gene-specific primers. In various embodiments, primers in the reagents are universal primers. Example universal primers include primers including at least 3 consecutive deoxythymidine nucleobases (e.g., oligo dT primer). In various embodiments, such primers in the reagents are reverse primers. In particular embodiments, primers in the reagents are only reverse primers and do not include forward primers.

In various embodiments, for targeted nucleic acid (e.g., targeted DNA or targeted RNA) sequencing, primers in the reaction mixture (e.g., reaction mixture 140 in FIG. 1B) include forward primers that are complementary to a forward target on a nucleic acid of interest (e.g., RNA or gDNA). In particular embodiments, the reaction mixture includes forward primers that are complementary to a forward target on a cDNA strand (generated from a RNA transcript) and further includes forward primers that are complementary to a forward target on gDNA. In various embodiments, primers in the reaction mixture are gene-specific primers that target a forward target of a gene of interest.

The number of forward or reverse primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. In various embodiments, genes of interest for either DNA-sequencing or RNA-sequencing include, but are not limited to: CCND3, CD44, CCND1, CD33, CDK6, CDK4, CDKN1B, CREB3L4, CDKN1A, CREBBP, CREB3L1, CREBS, CREB1, ELK1, FOS, FHL1, FASLG, GNG12, GSK3B, BAD, FOXO4, FOXO1, HIF1A, HSPB1, IKBKG, IRF9, BCL2, BCL2L11, MAP2K1 MAPK1, BCL2L1, MYB, NF1, NFKB1, MYC, PIK3CB, PIM1, PIAS1, PRKCB, PTEN, HSPA1A, HSPA2, IL2RB, IL2RA, SIRT1, NCL, RHOA, MCM4, NASP, SOS1, TCL1B, SOCS3, SOCS2, STAT4, STAT6, SRF, TP53, CASP9, CASP3, CASP8, UBB, MPRL16, MRPL21, FAM32A, ABCB7, PCBP1. EPS15, NRAS, RPS27A, AFF3, PAX3, CMTM6, RHOA, PIK3CA, MAP3K13, NSD1, PTPRK, CARD11, EGFR, EZH2, WRN, JAK2, GATA3, DKK1, POLA2, CCND1, ATM, ARHGEF12, KRAS, COL2A1, KMT2D, CLIP1, FLT3, BRCA2, BUB1B, PALB2, FANCA, NCOR1, ERBB2, KAT2A, RAB5C, METTL23, SRSF2, MFSD11, DNM2, CIC, BCR, MYH9, EP300, and SSX1.

In various embodiments, the primers used for targeted DNA or targeted RNA sequencing are cleavable primers. In various embodiments, the reaction mixture includes cleavable forward primers that are complementary to a forward target on a cDNA strand (generated from a RNA transcript) or includes cleavable forward primers that are complementary to a forward target on gDNA. In various embodiments, the reaction mixture includes cleavable forward primers that are complementary to a forward target on a cDNA strand (generated from a RNA transcript) and further includes cleavable forward primers that are complementary to a forward target on gDNA.

The cleavable primers used for targeted DNA or targeted RNA sequencing include a cleavage site. In various embodiments, the cleavable primers include a cleavage site located at the 5′ end or the 3′ end of the random primer. In various embodiments, the cleavable primers includes a cleavage site located within the cleavable primer. For example, the cleavable primer may include a constant region (also referred to as a PCR handle in FIG. 4B). Thus, the cleavable primer may include a cleavage site that is located between the constant region and the portion of the cleavable primer that hybridizes with the DNA or the cDNA sequence.

For whole transcriptome RNA sequencing, in various embodiments, the primers of the reagents (e.g., reagents 120 in FIG. 1B) can include a random primer sequence. In various embodiments, the random primer hybridizes with a sequence of reverse transcribed cDNA, thereby enabling priming off of the cDNA. In various embodiments, the reagents 120 includes various different random primers that enables priming off of all or a majority of cDNA generated from mRNA transcripts across the transcriptome. This enables the processing and analysis of mRNA transcripts across the whole transcriptome. In various embodiments, a random primer comprises a sequence of 5 nucleobases. In various embodiments, a random primer comprises a sequence of 6 nucleobases. In various embodiments, a random primer comprises a sequence of 9 nucleobases. In various embodiments, a random primer comprises a sequence of at least 5 nucleobases. In various embodiments, a random primer comprises a sequence of at least 6 nucleobases. In various embodiments, a random primer comprises a sequence of at least 9 nucleobases. In various embodiments, a random primer comprises a sequence of at least 6 nucleobases, at least 7 nucleobases, at least 8 nucleobases, at least 9 nucleobases, at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least 25 nucleobases, at least 26 nucleobases, at least 27 nucleobases, at least 28 nucleobases, at least 29 nucleobases, at least 30 nucleobases, at least 31 nucleobases, at least 32 nucleobases, at least 33 nucleobases, at least 34 nucleobases, or at least 35 nucleobases.

In various embodiments, random primers are cleavable primers. A random primer can include a cleavage site. In various embodiments, the random primer includes a cleavage site located at the 5′ end or the 3′ end of the random primer. In various embodiments, the random primer includes a cleavage site located within the random primer. For example, the random primer may include a constant region (also referred to as a FWD site in FIG. 4C). Thus, the random primer may include a cleavage site that is located between the constant region and the portion of the random primer that hybridizes with the cDNA sequence.

In various embodiments, primers of the reaction mixture, primers of the reagents, or primers of barcodes may be added to an emulsion in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the primers of the reaction mixture may be added in a separate step from the addition of a lysing agent (e.g., as exemplified in the two step workflow process shown in FIG. 1B).

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

Restriction Endonucleases

Also described herein are methods for processing products in the reaction mixture with restriction endonucleases prior to sequencing. Restriction endonucleases (or “restriction enzymes”) are enzymes that can be derived from bacteria and cleave double-stranded DNA (dsDNA) at specific recognition sequences (i.e., “cleavage sites”). In various embodiments, restriction endonuclease cleavage sites are palindromic. Restriction endonucleases cleavage sites can be 4 base pairs (bp), 5 bp, 6 bp, 7 bp, 8 bp, or 9 or more bp in length. In some embodiments, restriction endonucleases can leave single-strand nucleotide overhangs following cleavage. In some embodiments, restriction endonucleases can leave blunt ends (i.e., no nucleotide overhangs) following cleavage.

In some aspects described herein, restriction endonucleases can be selected for low frequencies of cleavage sites in the human genome. In some embodiments, selected restriction endonucleases can have 10,000 or fewer, 9,000 or fewer, 8,000 or fewer, 7,000 or fewer, 6,000 or fewer, 5,00 or fewer cleavage sites in the human genome. In some embodiments, selected restriction endonucleases can have 10,000 or fewer cleavage sites in the human genome. In some embodiments, selected restriction endonucleases can have 9,000 or fewer cleavage sites in the human genome. In some embodiments, selected restriction endonucleases can have 8,000 or fewer cleavage sites in the human genome. In some embodiments, selected restriction endonucleases can have 7,000 or fewer cleavage sites in the human genome. In some embodiments, selected restriction endonucleases can have 6,000 or fewer cleavage sites in the human genome. In some embodiments, selected restriction endonucleases can have 5,000 or fewer cleavage sites in the human genome.

In some aspects described herein, restriction endonucleases can be selected based on a higher frequency of cleavage sites in introns than in exons. In some embodiments, selected restriction endonucleases have 50% or more, 60% or more, 70% or more, 80% or more, or 90% or more frequency of genomic cleavage sites in introns. In some embodiments, selected restriction endonucleases have 50% or more frequency of genomic cleavage sites in introns. In some embodiments, selected restriction endonucleases have 60% or more frequency of genomic cleavage sites in introns. In some embodiments, selected restriction endonucleases have 70% or more frequency of genomic cleavage sites in introns. In some embodiments, selected restriction endonucleases have 80% or more frequency of genomic cleavage sites in introns. In some embodiments, selected restriction endonucleases have 90% or more frequency of genomic cleavage sites in introns.

In some aspects described herein, restriction endonucleases can have a high frequency of genomic cleavage sites in CpG islands. In some embodiments, selected restriction endonucleases can have 75% or more, 80% or more 85% or more, or 90% or more frequency of genomic cleavage sites in CpG islands. In some embodiments, selected restriction endonucleases can have 75% or more frequency of genomic cleavage sites in CpG islands. In some embodiments, selected restriction endonucleases can have 80% or more frequency of genomic cleavage sites in CpG islands. In some embodiments, selected restriction endonucleases can have 85% or more frequency of genomic cleavage sites in CpG islands. In some embodiments, selected restriction endonucleases can have 90% or more frequency of genomic cleavage sites in CpG islands.

In some embodiments, restriction endonucleases are selected from the list comprising AscI, NotI, AfeI, and SapI. In some embodiments, the selected restriction endonuclease is AscI. In some embodiments, the selected restriction endonuclease is NotI. In some embodiments, the selected restriction endonuclease is AfeI. In some embodiments, the restriction endonuclease is Sapl. In some embodiments, the selected restriction endonucleases include AscI and NotI. In some embodiments, the selected restriction endonucleases include AscI and AfeI. In some embodiments, the selected restriction endonucleases include AscI and SapI. In some embodiments, the selected restriction endonucleases include NotI and AfeI. In some embodiments, the selected restriction endonucleases include NotI and SapI. In some embodiments, the selected restriction endonucleases include AfeI and SapI. In some embodiments, the selected restriction endonucleases include AscI, NotI, and AfeI. In some embodiments, the selected restriction endonucleases include AscI, NotI, and SapI. In some embodiments, the selected restriction endonucleases include AscI, AfeI, and SapI. In some embodiments, the selected restriction endonucleases include NotI, AfeI, and SapI. In some embodiments, the selected restriction endonucleases include −AscI, NotI, AfeI, and SapI.

### Example System and/or Computer Embodiments

FIG. 7 depicts an example computing device (e.g., computing device 180 shown in FIG. 1A) for implementing system and methods described in reference to FIGS. 1-7. For example, the example computing device 180 is configured to perform the in silico steps of read alignment 215 and/or characterization 220. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

FIG. 7 illustrates an example computing device 180 for implementing system and methods described in FIGS. 1-6. In some embodiments, the computing device 180 includes at least one processor 802 coupled to a chipset 804. The chipset 804 includes a memory controller hub 820 and an input/output (I/O) controller hub 822. A memory 806 and a graphics adapter 812 are coupled to the memory controller hub 820, and a display 818 is coupled to the graphics adapter 812. A storage device 808, an input interface 814, and network adapter 816 are coupled to the I/O controller hub 822. Other embodiments of the computing device 180 have different architectures.

The storage device 808 is a non-transitory computer-readable storage medium such as a hard drive, compact disk read-only memory (CD-ROM), DVD, or a solid-state memory device. The memory 806 holds instructions and data used by the processor 802. The input interface 814 is a touch-screen interface, a mouse, track ball, or other type of input interface, a keyboard, or some combination thereof, and is used to input data into the computing device 180. In some embodiments, the computing device 180 may be configured to receive input (e.g., commands) from the input interface 814 via gestures from the user. The graphics adapter 812 displays images and other information on the display 818. For example, the display 818 can show metrics pertaining to the generated libraries (e.g., DNA or RNA libraries) and/or any characterization of single cells. The network adapter 816 couples the computing device 180 to one or more computer networks.

The computing device 180 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 808, loaded into the memory 806, and executed by the processor 802.

The types of computing devices 180 can vary from the embodiments described herein. For example, the computing device 180 can lack some of the components described above, such as graphics adapters 812, input interface 814, and displays 818. In some embodiments, a computing device 180 can include a processor 802 for executing instructions stored on a memory 806.

The methods of aligning sequence reads and characterizing libraries and/or cells can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Example Kit Embodiments

Also provided herein are kits for performing single cell analysis of RNA transcripts and genomic DNA of individual or populations of cells. The kits may include one or more of the following: fluids for forming emulsions (e.g., carrier phase, aqueous phase), barcoded beads, micro fluidic devices for processing single cells, reagents for lysing cells and releasing cell analytes, reaction mixtures for performing nucleic acid amplification reactions, and instructions for using any of the kit components according to the methods described herein. In particular embodiments, the kits include cleavable primers as well as agents, such as restriction endonucleases, for cleaving the cleavable primers for cleaning up misprimed amplicons.

### Additional Embodiments

Detection of target nucleic acid is useful to many applications including disease detection. The nucleic acid may be derived from a single cell, a molecule or a protein. An exemplary method for detecting nucleic acid according to one embodiment of the disclosure includes several steps. These steps may be implemented sequentially or non-sequentially. further, one or more of the steps may be implemented optionally.

The first step may comprise selecting a target nucleic acid sequence of interest in an individual cell. The target nucleic acid sequence can be complementary to an mRNA that has a corresponding genomic DNA comprising one or more cleavage sequence. The target nucleic acid may optionally exclude endonuclease recognition and cleavage sequences. In one embodiment, the DNA cleavage sequence comprises a restriction enzyme cleavage recognition sequence. In certain embodiments, the restriction enzyme is selected from those having a low frequency in the human genome. In one embodiment, the restriction enzyme is selected such that the cleavage sites are in introns and human genome exonic regions are avoided. In another embodiment, the restriction enzyme is selected such that the cleavage sites are in GC rich regions in the human genome. In still another embodiment, the restriction enzyme is selected from AscI, NotI, AfeI, SapI or combinations thereof. In yet another embodiment, an additional step of performing a restriction enzyme digest may be performed.

In certain embodiments, the DNA cleavage sequence comprises a modification in the DNA that makes it susceptible to cleavage. In some embodiments, the modification in a DNA (oligonucleotide primer) is selected from Iso-dC and Iso-dG modified oligos, methylated oligos (5-methyl dC) that can be digested with enzymes that can digest hemimethylated DNA. dUTP modified oligonucleotides that can be digested with the USER enzyme, Uracil DNA glycosylase (UDG) and the DNA glycosylase-lyase Endonuclease VIII.

A second step comprises providing a sample having a plurality of individual single cells. The sample may be obtained in conventional methods. Once obtained, the one or more individual cell(s) are encapsulated in a reaction mixture comprising a protease. In certain embodiments, the reaction mixture may comprise a droplet. That is, the reaction mixture including the one or more cells and the protease may be encapsulated in a droplet or a plurality of droplets. In one embodiment, the droplet itself may be encapsulated in another droplet. The droplets may define an aqueous emulsion in a non-aqueous solution. In certain embodiments, the analysis of both DNA and RNA occurs in a single drop. In one embodiment, the reaction mixture further comprises a polymerase for synthesizing cDNA from mRNA, DNA extension, hybridization, capture or ligation.

In some embodiments, the protease is cytolytic. In an exemplary embodiment, the protease is selected from proteinase K.

The third step comprises incubating the encapsulated cell with the protease in the drop to produce a cell lysate. The incubation may be done isothermally. In one embodiment, the incubation period includes thermocycling.

The fourth step comprises providing an affinity reagent that binds the target nucleic acid. The affinity reagent may have an oligonucleotide comprising a first nucleic acid barcode that is an indicator of the affinity reagent binding the target nucleic acid. The first nucleic acid barcode may define an identification sequence. In one embodiment, the affinity reagent comprises an antibody or an antigen binding antibody fragment.

The fifth step comprises contacting the affinity reagent to the target nucleic acid under conditions sufficient for binding of the affinity reagent to the target nucleic acid to form an affinity reagent bound target nucleic acid.

The sixth step comprises adding the affinity reagent bound target nucleic acid target to the reaction mixture.

The seventh step may be optional. The seventh step includes providing an oligonucleotide comprising a second nucleic acid barcode. The second nucleic acid barcode serves as an indicator of a particular reaction mixture in which the affinity reagent bound molecular target is encapsulated.

The eight step comprises adding at least one oligonucleotide primer pair to the reaction mixture. In one application one or both of the oligonucleotides of the pair include a first nucleic acid barcode that is an indicator of the affinity reagent binding the target nucleic acid. Additionally, one or both of the oligonucleotides of the pair comprise a restriction endonuclease cleavage site.

The ninth step comprises performing cleavage of the DNA cleavage sequence to remove unwanted genomic DNA.

The tenth step includes amplifying the target nucleic acids remaining after the cleavage (restriction enzyme digest).

The eleventh steps comprises determining the identity of the target nucleic acids by sequencing the first bar code and second bar code and wherein detection is enhanced by reducing the detection of non-target nucleic acids. The non-target nucleic acid may comprise corresponding genomic DNA nucleic acids.

In one embodiment, the enhanced detection of a target nucleic acid sequence is at least partially selective for the detection of mRNA. In another embodiment, the reaction mixture is aqueous, an aqueous emulsion in oil, or an aqueous suspension in oil. In certain applications, the reaction mixture is in a single drop. In still another embodiment, the enhanced detection of a target nucleic acid provides increased specificity. In yet another embodiment, the enhanced detection of a target nucleic acid has a decreased incidence of false positives.

Another embodiment of the disclosure relates to a primer design for selective detection of nucleic acid(s) in a mixture of DNA and mRNA. In an implementation, a method of primer design for selective detection of nucleic acids in a sample comprising both the genomic DNA and mRNA. The comprises, the optional steps of: (i) selecting a target nucleic acid sequence of interest in an individual cell, wherein the target nucleic acid sequence is complementary to a mRNA of interest that has a corresponding genomic DNA, wherein the target nucleic acid sequence comprises one or more cleavage sequence within the corresponding genomic DNA, (ii) providing at least one set of oligonucleotide primers where one or more of the primers comprises a cleavage sequence in the genomic DNA corresponding to the target sequence, wherein the use of the oligonucleotide primers comprising a cleavage sequence in an amplification reaction allows the selective detection of a target mRNA over its corresponding genomic DNA in a sample comprising both the genomic DNA and mRNA.

In another aspect, the disclosure relates to a microfluidic system for implementing the disclosed principles. The microfluidic system comprises means for selecting a target nucleic acid sequence of interest in an individual cell, wherein the target nucleic acid sequence is complementary to a mRNA that has a corresponding genomic DNA comprising one or more cleavage sequence; means for providing a sample having a plurality of individual single cells; encapsulating one or more individual cell(s) in a reaction mixture comprising a protease; means for incubating the encapsulated cell with the protease in the drop to produce a cell lysate; means for providing an affinity reagent that binds the target nucleic acid, said affinity reagent having an oligonucleotide comprising a first nucleic acid barcode (identification sequence) that is an indicator of the affinity reagent binding the target nucleic acid; means for contacting the affinity reagent to the target nucleic acid under conditions sufficient for binding of the affinity reagent to the target nucleic acid to form an affinity reagent bound target nucleic acid; means for adding the affinity reagent bound target nucleic acid target to the reaction mixture; means for, optionally, providing an oligonucleotide comprising a second nucleic acid barcode, wherein the second nucleic acid barcode serves as an indicator of a particular reaction mixture in which the affinity reagent bound molecular target is encapsulated; means for adding at least one oligonucleotide primer pair to the reaction mixture, wherein one or both of the oligonucleotides of the pair comprise a first nucleic acid barcode that is an indicator of the affinity reagent binding the target nucleic acid, and where one or both of the oligonucleotides of the pair comprise a restriction endonuclease cleavage site; means for performing cleavage of the DNA cleavage sequence to remove unwanted genomic DNA; means for amplifying the target nucleic acids remaining after the cleavage (restriction enzyme digest); and means for determining the identity of the target nucleic acids by sequencing the first bar code and second bar code and wherein detection is enhanced by reducing the detection of non-target (corresponding genomic DNA) nucleic acids.

It is noted that disclosed are merely illustrative and non-limiting of the disclosed principles. Other applicants of the disclosed principles can be made without departing from the spirit of the disclosed principles.

Disclosed herein is a method for enhanced detection of a target nucleic acid from a single cell, the method comprising, independent of order presented, the following steps: selecting a target nucleic acid sequence of interest in an individual cell, wherein the target nucleic acid sequence is complementary to a mRNA that has a corresponding genomic DNA comprising one or more cleavage sequence (restriction endonuclease recognition and cleavage sequences); providing a sample having a plurality of individual single cells; encapsulating one or more individual cell(s) in a reaction mixture comprising a protease; incubating the encapsulated cell with the protease in the drop to produce a cell lysate; providing an affinity reagent that binds the target nucleic acid, said affinity reagent having an oligonucleotide comprising a first nucleic acid barcode (identification sequence) that is an indicator of the affinity reagent binding the target nucleic acid; contacting the affinity reagent to the target nucleic acid under conditions sufficient for binding of the affinity reagent to the target nucleic acid to form an affinity reagent bound target nucleic acid; adding the affinity reagent bound target nucleic acid target to the reaction mixture; optionally, providing an oligonucleotide comprising a second nucleic acid barcode, wherein the second nucleic acid barcode serves as an indicator of a particular reaction mixture in which the affinity reagent bound molecular target is encapsulated; adding at least one oligonucleotide primer pair to the reaction mixture, wherein one or both of the oligonucleotides of the pair comprise a first nucleic acid barcode that is an indicator of the affinity reagent binding the target nucleic acid, and where one or both of the oligonucleotides of the pair comprise a restriction endonuclease cleavage site; performing cleavage of the DNA cleavage sequence to remove unwanted genomic DNA; amplifying the target nucleic acids remaining after the cleavage (restriction enzyme digest); and determining the identity of the target nucleic acids by sequencing the first bar code and second bar code and wherein detection is enhanced by reducing the detection of non-target (corresponding genomic DNA) nucleic acids.

In various embodiments, the enhanced detection of a target nucleic acid sequence is at least partially selective for the detection of mRNA. In various embodiments, the reaction mixture is aqueous, an aqueous emulsion in oil, or an aqueous suspension in oil. In various embodiments, the reaction mixture is in a single drop. In various embodiments, the protease is cytolytic. In various embodiments, the protease is selected from proteinase K. In various embodiments, the analysis of both DNA and RNA occurs in a single drop. In various embodiments, the analysis of cellular DNA and RNA from an individual cell occurs in a single drop.

In various embodiments, the DNA cleavage sequence comprises a restriction enzyme cleavage recognition sequence. In various embodiments, the restriction enzyme is selected from those having a low frequency in the human genome. In various embodiments, the restriction enzyme is selected such that the cleavage sites are in introns and human genome exonic regions are avoided. In various embodiments, the restriction enzyme is selected such that the cleavage sites are in GC rich regions in the human genome.

In various embodiments, the restriction enzyme is selected from AscI, NotI, AfeI or SapI. In various embodiments, the method further comprises the step of performing a restriction enzyme digest. In various embodiments, the DNA cleavage sequence comprises a modification in the DNA that makes it susceptible to cleavage. In various embodiments, the modification in a DNA (oligonucleotide primer) is selected from Iso-dC and Iso-dG modified oligos, methylated oligos (5-methyl dC) that can be digested with enzymes that can digest hemimethylated DNA. dUTP modified oligonucleotides that can be digested with the USER enzyme, Uracil DNA glycosylase (UDG) and the DNA glycosylase-lyase Endonuclease VIII.

In various embodiments, the enhanced detection of a target nucleic acid has an increased specificity. In various embodiments, the enhanced detection of a target nucleic acid has a decreased incidence of false positives. In various embodiments, the affinity reagent comprises an antibody or an antigen binding antibody fragment. In various embodiments, the reaction mixture further comprises a polymerase for synthesizing cDNA from mRNA, DNA extension, hybridization, capture or ligation.

Additionally disclosed herein is a method of primer design for selective detection of nucleic acids in a sample comprising both the genomic DNA and mRNA, the method comprising: i) selecting a target nucleic acid sequence of interest in an individual cell, wherein the target nucleic acid sequence is complementary to a mRNA of interest that has a corresponding genomic DNA, wherein the target nucleic acid sequence comprises one or more cleavage sequence within the corresponding genomic DNA, ii) providing at least one set of oligonucleotide primers where one or more of the primers comprises a cleavage sequence in the genomic DNA corresponding to the target sequence, wherein the use of the oligonucleotide primers comprising a cleavage sequence in an amplification reaction allows the selective detection of a target mRNA over its corresponding genomic DNA in a sample comprising both the genomic DNA and mRNA.

### EXAMPLES

### Example 1

### Single Cell Sequencing in a Droplet with Enhanced Detection

Cells were loaded into a microfluidic cartridge and co-encapsulated into droplets with a lysis buffer containing protease and mild detergent. The oligonucleotides used at this stage could contain the restriction sites or modified oligos at different concentration ranging from 1 pM to 1 mM. Droplets were incubated in a thermal cycler for 1 h at 50° C. to digest all cellular proteins, followed by 10 min at 80° C. to heat-inactivate the protease. Lysed cells in droplets were transferred to the barcoding module of the microfluidic cartridge in addition to polymerase mix, the modified reverse primer pool, barcoded hydrogel beads, and oil for droplet generation. The oligonucleotides used at this stage could contain the restriction sites or modified oligos at different concentration ranging from 1 pM to 1 mM. The droplets were placed under a UV lamp (Analytik Jena, Blak-Ray XX15L) for 8 min to cleave the single-stranded PCR primers containing unique cell barcodes from the hydrogel beads. To amplify DNA, RNA or protein, droplets were thermal cycled using the following program: 95° C. for 10 m; 20 cycles of (95° C. for 30 s, 72° C. for 10 s, 61° C. for 4 min, 72° C. for 30 s); 72° C. for 2 min; 4° C. hold. Recovery and cleanup of single-cell libraries proceeded according to the Mission Bio V2 protocol with additional modifications for oligo digestion. The 8 PCR tubes containing barcoded droplets were pooled as pairs and treated with Mission Bio Extraction Agent. Water was added to each tube and the aqueous fraction transferred to a new 1.5 mL DNA LoBind tube. Restriction enzymes to target the oligo restriction sites or modified oligos were added at different concentration ranging from 1 pM to 1 mM and incubated at temperature ranges from 4 C to 99 C for 1 min-24 h incubation time. Ampure XP beads (Beckman Coulter, cat. no. A63881) were added at different ratio beads:PCR product for size selection of the RNA, DNA or protein products. PCR was performed on the purified DNA, RNA or Protein products to produce sequencing libraries. For each tube of purified DNA panel, 50 μL reactions were prepared containing 4 ng of barcoded product in 15 μL water, 25 μL Mission Bio Library Mix, and 5 μL each of custom P5 and Nextera P7 primers (N7XX), both at 4 μM stock concentration. The reactions were thermal cycled using the following program: 95° C. for 3 min; 10 cycles of (98° C. for 20 s, 62° C. for 20 s, 72° C. for 45 s); 72° C. for 2 min; 4° C. hold. Following amplification, the DNA, RNA or protein libraries were cleaned with different ranges of Ampure XP beads and eluted in water.

Table 1 shows results of library structure and genome mapping of reads. These results show greater than 60% improved library structure and mapping of reads to the genome when enzymatic clean-up is performed on amplicons prior to sequencing.

TABLE 1
Library structure and genome mapping of reads

| | | % Correct | % Mapping |
|---|---|---|---|
| | | Library Structure | to Genome |
| Standard | S1 | 34 | 13 |
| | S2 | 37 | 15 |
| | S3 | 28 | 13 |
| | S4 | 46 | 18 |
| | S5 | 37 | 16 |
| | S.Avg | 36 | 15 |
| Enzymatic Clean-Up | E1 | 58 | 24 |
| | E2 | 51 | 23 |
| | E3 | 60 | 23 |
| | E4 | 64 | 27 |
| | E5 | 62 | 29 |
| | E.Avg | 59 | 25 |

These results indicate that enzymatic clean-up is an effective method of improving sequencing quality from single-cell samples.

What is claimed is:

1. A method for determining the presence or absence of a target nucleic acid from a single cell, the method comprising:
a. obtaining a cell lysate from a single cell in a reaction mixture droplet;
b. adding oligonucleotide primers to the reaction mixture droplet, wherein the oligonucleotide primers comprise restriction endonuclease cleavage sites;
c. catalyzing a nucleic acid amplification reaction using the primers to produce an amplicon from a target nucleic acid molecule, if present, in the single cell;
d. adding restriction endonucleases to the reaction mixture droplet and incubating to cleave, if present, nucleic acid amplicons that have been misprimed by the oligonucleotide primers;
e. sequencing the remaining amplicons; and
f. determining the presence or absence of the target nucleic acid from the single cell based on the presence or absence of the target sequence in the determined sequences of the amplicons.

2. The method of claim 1, wherein the nucleic acid amplicons comprise misprimed DNA that have been misprimed by oligonucleotide primers.

3. The method of claim 1 or 2, wherein the target nucleic acid sequence is complementary to an mRNA that corresponds to the DNA, wherein the DNA comprises a restriction endonuclease cleavage sequence.

4. The method of any one of claims 1-3, wherein the oligonucleotide primers are DNA primers.

5. The method of any one of claims 1-4, wherein one of the oligonucleotide primers comprises a forward primer that is complementary to the target nucleic acid sequence.

6. The method of claim 5, wherein the forward primer is a gene specific primer.

7. The method of claim 5 or 6, wherein the one of the oligonucleotide primers further comprises a constant region.

8. The method of claim 7, wherein a restriction endonuclease cleavage site is located between the constant region and the forward primer.

9. The method of any one of claims 1-8, wherein the DNA is genomic DNA.

10. The method of any one of claims 1-8, wherein the DNA is an oligonucleotide sequence corresponding to an affinity reagent.

11. The method of claim 10, wherein the oligonucleotide sequence was previously conjugated to the affinity reagent.

12. The method of any one of claims 1-4, wherein one of the oligonucleotide primers comprises a random primer that is complementary to the target nucleic acid sequence.

13. The method of claim 12, wherein the one of the oligonucleotide primers further comprises a constant region.

14. The method of claim 13, wherein a restriction endonuclease cleavage site is located between the constant region and the random primer.

15. The method of claim 1, wherein the nucleic acid amplicons comprise misprimed cDNA generated from RNA of the single cell, wherein the misprimed cDNA have been misprimed by oligonucleotide primers.

16. The method of claim 1 or 15, wherein the target nucleic acid sequence is a gDNA that corresponds to the misprimed cDNA, wherein the misprimed cDNA comprises one or more restriction endonuclease cleavage sequences.

17. The method of any one of claim 1 or 15-16, wherein the oligonucleotide primers are DNA primers.

18. The method of any one of claim 1 or 15-17, wherein one of the oligonucleotide primers comprises a forward primer that is complementary to the target nucleic acid sequence.

19. The method of claim 18, wherein the forward primer is a gene specific primer.

20. The method of claim 18 or 19, wherein the one of the oligonucleotide primers further comprises a constant region.

21. The method of claim 20, wherein a restriction endonuclease cleavage site is located between the constant region and the forward primer.

22. The method of any one of claims 1-21, wherein the restriction endonucleases have low frequencies of cleavage sites in gDNA.

23. The method of any one of claims 1-22, wherein the restriction endonucleases have higher frequencies of genomic cleavage sites in introns than in exons.

24. The method of claim 23, wherein the restriction endonucleases have 50% or more, 60% or more, 70% or more, 80% or more, or 90% or more frequency of genomic cleavage sites in introns.

25. The method of any one of claims 1-24, wherein the restriction endonucleases have a high frequency of cleavage sites in the human genome in CpG islands.

26. The method of claim 25, wherein the restriction endonucleases have 75% or more, 80% or more 85% or more, or 90% or more cleavage sites in the human genome in CpG islands.

27. The method of any one of claims 1-26, wherein the restriction endonucleases are selected from AscI, NotI, AfeI, and SapI.

28. The method of any one of claims 1-27, wherein the determination of the presence or absence of the target nucleic has increased specificity compared to detection in samples where restriction endonucleases are not added.

29. The method of claim 28, wherein the determination of the presence or absence of the target nucleic acid has 50% or more, 60% or more, 70% or more, 80% or more, or 90% or more increased specificity compared to detection in samples where restriction endonucleases are not added.

30. The method of any one of claims 1-29, wherein the determination of the presence or absence of the target nucleic has a decreased incidence of false positives compared to detection in samples where restriction endonucleases are not added.

31. The method of claim 30, wherein the determination of the presence or absence of the target nucleic acid has 50% or more, 60% or more, 70% or more, 80% or more, or 90% or more decreased incidence of false positives compared to detection in samples where restriction endonucleases are not added.

32. The method of any one of claims 1-31, further comprising adding oligonucleotide barcodes to the reaction mixture droplet, the oligonucleotide barcodes specific for the reaction mixture droplet.

33. The method of claim 32, wherein the sequencing comprises identification of target nucleic acids by identification of the oligonucleotide barcode.

34. The method of claim 32, wherein the sequencing comprises:
sequencing a nucleic acid lacking an oligonucleotide barcode; and
removing the sequenced nucleic acid for further analysis.

35. The method of claim 34, wherein the nucleic acid lacking the oligonucleotide barcode was previously a nucleic acid amplicon that was misprimed by an oligonucleotide primer and cleaved by a restriction endonuclease.

36. The method of any one of claims 1-35, wherein the reaction mixture droplet is an aqueous solution, an aqueous emulsion in oil, or an aqueous suspension in oil.

37. The method of any one of claims 1-36, wherein the reaction mixture droplet comprises a DNA-modifying enzyme for synthesizing cDNA from RNA, DNA extension, hybridization, capture, or ligation.

38. The method of any one of claims 1-37, wherein obtaining the cell lysate from the single cell in the reaction mixture droplet comprises exposing the single cell in the reaction mixture droplet to a protease.

39. The method of claim 38, wherein the protease is proteinase K.
